(19) United States
(12) Patent Application Publication
(10) Pub. No.: US 2024/0075080 A1
(43) Pub. Date: Mar. 7, 2024

(54) USE OF LACTIC ACID BACTERIA TO INHIBIT METHANOGEN GROWTH OR REDUCE METHANE EMISSIONS

(71) Applicants: Graeme Trevor ATTWOOD, Hamilton (NZ); Laureen CROUZET, Hamilton (NZ); Shalome Anitta BASSETT, Auckland (NZ); James William DEKKER, Auckland (NZ); Jeremy Paul HILL, Auckland (NZ); Fonterra Co-Operative Group Limited, Auckland (NZ); Agresearch Limited, Hamilton (NZ)

(72) Inventors: Graeme Trevor Attwood, Hamilton (NZ); Laureen Crouzet, Hamilton (NZ); Shalome Anitta Bassett, Auckland (NZ); James William Dekker, Auckland (NZ); Jeremy Paul Hill, Auckland (NZ)

(21) Appl. No.: 18/258,950

(22) PCT Filed: Dec. 22, 2021
(86) PCT No.: PCT/IB2021/062163

§ 371 Date: Jun. 22, 2023

(30) Foreign Application Priority Data
Dec. 22, 2020 (AU) 2020904788

(51) Int. Cl. A61K 35/747; A23K 10/18; A23K 50/10; A61P 31/04

(52) U.S. Cl. CPC A61K 35/747; A23K 10/18; A23K 50/10; A61P 31/04

(57) ABSTRACT

This invention relates to use of a strain of lactic acid bacteria for inhibiting the growth of methane-producing bacteria and/or archaea in the forestomach of ruminant animals, reducing the ability of the rumen microbiome to produce methane, reducing ruminal methane production and/or for improving feed efficiency, milk production, and/or body weight or body composition of a ruminant animal. Ruminant feed compositions are also provided.

### TECHNICAL FIELD

This invention relates the use of a strain of lactic acid bacteria for inhibiting the growth of methane-producing bacteria and/or archaea in the forestomach of ruminant animals, reducing the ability of the rumen microbiome to produce methane, reducing methane production and/or for improving feed efficiency, milk production, and/or body weight or body composition of a ruminant animal. Ruminant feed compositions are also provided.

### BACKGROUND

Methane is a potent greenhouse gas, absorbing infrared radiation much more efficiently than CO₂ and having a warming potential ˜86 times larger than its mass equivalent of CO₂ on a 20-year timescale (IPCC, 2014). While methane is a relatively low proportion of anthropogenic greenhouse gas emissions, it nevertheless is a significant contributor to climate change.

A major source of methane emissions is the fermentation of organic matter by methanogenic bacteria and archaea. One prevalent source of anthropogenic methane emissions is in agriculture, where methane is produced by enteric fermentation in the digestive tract of ruminants, and from manure. These sources accounted for ˜30% of total global anthropogenic methane emissions in 2017 (Jackson et al., 2020). In addition, not only does methanogenesis in ruminants result in greenhouse gas emissions, but it is also energetically wasteful to the animal. It has long been recognised that methane production in ruminants dramatically impacts the efficiency with which these animals convert feed into metabolic energy. This decrease in efficiency results because methane represents a caloric loss to the ruminant of approximately 5-10% of its total caloric intake.

Thus, there remains a need for methods and compositions useful for inhibiting the growth of methane-producing bacteria and/or archaea in the forestomach of ruminant animals, reducing the ability of the rumen microbiome to produce methane, and/or for reducing methane emissions by ruminant animals. Methods and compositions for increasing feed efficiency, increasing milk or meat production, and/or increasing body weight or improving body composition of ruminant animals are also desirable.

It is an object of this invention to go some way towards achieving one or more of these desiderata, or at least to offer the public a useful choice.

### SUMMARY OF THE INVENTION

In a first aspect the invention provides a method for inhibiting the growth of methane-producing bacteria and/or archaea in the forestomach of ruminant animals, or for reducing the ability of the rumen microbiome to produce methane, wherein the method comprises administering to a ruminant animal an effective amount of Lacticaseibacillus rhamnosus strain HN001, AGAL deposit number NM97/09514 dated 18 Aug. 1997, or a derivative thereof.

In a second aspect the invention provides a method for reducing ruminal methane production by a ruminant animal, wherein the method comprises administering to the animal an effective amount of Lacticaseibacillus rhamnosus strain HN001, AGAL deposit number NM97/09514 dated 18 Aug. 1997, or a derivative thereof.

In a third aspect the invention provides a method for increasing feed efficiency in a ruminant animal, wherein the method comprises administering to the animal an effective amount of Lacticaseibacillus rhamnosus strain HN001, AGAL deposit number NM97/09514 dated 18 Aug. 1997, or a derivative thereof.

In a fourth aspect, the invention provides a method for improving the absorptive capacity of the forestomach, for example increasing the absorptive capacity for volatile fatty acids (VFAs), wherein the method comprises administering to the animal an effective amount of Lacticaseibacillus rhamnosus strain HN001, AGAL deposit number NM97/09514 dated 18 Aug. 1997, or a derivative thereof.

In a fifth aspect, the invention provides a method for enhancing the physical and/or functional development of the rumen, or other chambers of the forestomach, in a ruminant, for example a young ruminant, for example a young ruminant prior to weaning, wherein the method comprises administering to the animal an effective amount of Lacticaseibacillus rhamnosus strain HN001, AGAL deposit number NM97/09514 dated 18 Aug. 1997, or a derivative thereof.

In one embodiment, the method enhances anatomical development of the rumen. For example, the method enhances development of rumen epithelium and/or muscularisation, for example increasing growth of rumen mass, growth of rumen papillae, increase in papillae density, for example dorsal papillae density, and/or total surface area of the ruminal wall in the animal.

In one embodiment, the method enhances rumen weight, ruminal wall thickness, or density of rumen papillae per cm² of ruminal wall, for example compared to an untreated animal.

In one embodiment, the method increases rumen papillae length, width, and/or surface area. For example, in some embodiments, the method increases rumen papillae length, width, and/or surface area to at least 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, 1.19, 1.20, 1.22, 1.24, 1.26, 1.28, 1.30, 1.32, 1.34, 1.36, 1.38, or 1.40 times that of an untreated animal.

In one embodiment, the method enhances functional achievement of the rumen, or promotes maturation of the forestomach. For example, the method stimulates rumination, enhances dry matter intake (DMI), enhances absorptive ability and/or promotes maturation towards a mature physiology.

In some embodiments, the method inhibits the growth of a hydrogenotrophic methanogen in the forestomach of the animal. In one embodiment, the method inhibits the growth of a methanogen from the genus Methanobrevibacter in the forestomach of the animal.

In some embodiments, the L. rhamnosus HN001 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or intra-ruminal product, e.g., a bolus.

In some embodiments, the L. rhamnosus HN001 or derivative thereof is administered in drinking water, milk, milk powder, milk replacement, milk fortifier, whey, whey powder, Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

In some embodiments, the method comprises administering to the animal the L. rhamnosus HN001 in an amount of from 10⁴ to 10¹³ colony forming units per kilogram of dry weight carrier feed. In one embodiment, the method comprises administering to the animal the L. rhamnosus HN001 in an amount from 10⁸ to 10¹² colony forming units per kilogram of dry weight carrier feed.

In some embodiments, the derivative of L. rhamnosus strain HN001 is a cell lysate of the strain, a cell suspension of the strain, a metabolite of the strain, a culture supernatant of the strain, or killed L. rhamnosus HN001.

In some embodiments, the method comprises further administering at least one microorganism of a different species or strain, a vaccine that inhibits methanogens or methanogenesis, and/or a natural or chemically-synthesised inhibitor of methanogenesis and/or methanogen inhibitor. An example of a useful inhibitor of methanogenesis is bromoform, which works by inhibiting the efficiency of the methyltransferase enzyme by reacting with the reduced vitamin B12 cofactor required for the penultimate step of methanogenesis.

In one embodiment, the method comprises further administering at least one microorganism of a different species or strain, a vaccine that inhibits methanogens or methanogenesis, and/or a natural or chemically-synthesised inhibitor of methanogenesis and/or methanogen inhibitor that targets a methanogen that is not Methanobrevibacter, for example, a methylotrophic methanogen such as a methanogen from the genus Methanosphaera or the order Methanomassiliicoccale.

In some embodiments, the L. rhamnosus HN001 or derivative thereof is administered separately, simultaneously or sequentially with one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

In some embodiments, the method additionally enhances the growth or productivity of the animal, for example the method increases the yield of milk and/or milk components produced from the ruminant animal.

In some embodiments, the method additionally increases the body weight and/or improves body composition, such as altering the muscle to fat ratio, of the ruminant animal.

In some embodiments, the ruminant animal is a bovine, goat, sheep, bison, yak, water buffalo, deer, camel, alpaca, llama, wildebeest, antelope, or nilgai. In one embodiment, the ruminant animal are cattle or sheep. In one embodiment, the ruminant animal are cattle. In one embodiment, the ruminant animal is a lactating animal. In an alternative embodiment, the ruminant animal is a pre-weaning animal, such as a calf or a lamb.

In a sixth aspect, the invention provides a ruminant feed composition for inhibiting the growth of methane-producing bacteria and/or archaea in the forestomach of ruminant animals, reducing the ability of the rumen microbiome to produce methane, reducing ruminal methane production by a ruminant animal, or increasing feed efficiency in a ruminant animal, the feed composition comprising Lacticaseibacillus rhamnosus strain HN001, AGAL deposit number NM97/09514 dated 18 Aug. 1997, or a derivative thereof.

In some embodiments, the ruminant feed composition is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, lick block, or molasses.

In some embodiments, the ruminant feed composition further comprises at least one microorganism of a different species or strain, a vaccine that inhibits methanogens or methanogenesis, and/or a natural or chemically-synthesised inhibitor of methanogenesis and/or methanogen inhibitor such as bromoform.

In one embodiment, the at least one microorganism of a different species or strain, a vaccine that inhibits methanogens or methanogenesis, and/or a natural or chemically-synthesised inhibitor of methanogenesis and/or methanogen inhibitor targets a methanogen that is not Methanobrevibacter, for example, a methylotrophic methanogen such as a methanogen from the genus Methanosphaera or the order Methanomassiliicoccale.

In some embodiments, the ruminant feed composition further comprises one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

In a further aspect, the invention provides a method for inhibiting the growth of methane-producing bacteria and/or archaea in the forestomach of a ruminant animal, and/or reducing the ability of the rumen microbiome to produce methane, said method comprising the step of administering to said animal a ruminant feed composition according to the sixth aspect.

In a further aspect, the invention provides a method for reducing ruminal methane production by a ruminant animal, said method comprising the step of administering to said animal a ruminant feed composition according to the sixth aspect.

In a further aspect, the invention provides a method for increasing feed efficiency in a ruminant animal, said method comprising the step of administering to said animal a ruminant feed composition according to the sixth aspect.

In a further aspect, the invention provides a method for enhancing the growth and/or productivity in a ruminant animal, said method comprising the step of administering to said animal a ruminant feed composition according to the sixth aspect.

In a further aspect, the invention provides a method for increasing the yield of milk and/or milk components produced from a ruminant animal, said method comprising the step of administering to said animal a ruminant feed composition according to the sixth aspect.

In a further aspect, the invention provides a method for improving the body weight or body composition of a ruminant animal, said method comprising the step of administering to said animal a ruminant feed composition according to the sixth aspect.

In a further aspect, the invention provides a use of Lacticaseibacillus rhamnosus strain HN001, AGAL deposit number NM97/09514 dated 18 Aug. 1997, or a derivative thereof, for the manufacture of a composition for inhibiting the growth of methane-producing bacteria and/or archaea in the forestomach of ruminant animals, reducing the ability of the rumen microbiome to produce methane, reducing ruminal methane production by a ruminant animal, increasing feed efficiency in a ruminant animal, enhancing the growth and/or productivity in a ruminant animal, increasing the yield of milk and/or milk components produced from a ruminant animal, or improving the body weight and/or body composition of a ruminant animal.

In some embodiments, the composition is or comprises a ruminant feed composition according to the sixth aspect.

In a further aspect, the invention provides Lacticaseibacillus rhamnosus strain HN001, AGAL deposit number NM97/09514 dated 18 Aug. 1997, or a derivative thereof, for use in inhibiting the growth of methane-producing bacteria and/or archaea in the forestomach of ruminant animals, reducing the ability of the rumen microbiome to produce methane, reducing ruminal methane production by a ruminant animal, increasing feed efficiency in a ruminant animal, enhancing the growth and/or productivity in a ruminant animal, increasing the yield of milk and/or milk components produced from a ruminant animal, or improving the body weight and/or body composition of a ruminant animal.

In a further aspect, the invention provides a method for reducing methane emissions by a ruminant animal, wherein the method comprises administering to the animal an effective amount of Lacticaseibacillus rhamnosus strain HN001, AGAL deposit number NM97/09514 dated 18 Aug. 1997, or a derivative thereof.

In a further aspect, the invention provides a ruminant feed composition for inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, enhancing the growth and/or productivity in a ruminant animal, increasing the yield of milk and/or milk components produced from a ruminant animal, or improving the body weight and/or body composition of a ruminant animal, the feed composition comprising Lacticaseibacillus rhamnosus strain HN001 or a derivative thereof.

In a further aspect, the invention provides a method for reducing methane emissions by a ruminant animal, said method comprising the step of administering to said animal a ruminant feed composition according to the above aspect.

In a further aspect, the invention provides use of Lacticaseibacillus rhamnosus strain HN001, AGAL deposit number NM97/09514 dated 18 Aug. 1997, or a derivative thereof for the manufacture of a composition for inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, increasing the yield of milk and/or milk components produced from a ruminant animal, or improving the body weight and/or body composition of a ruminant animal.

In a further aspect, the invention provides Lacticaseibacillus rhamnosus strain HN001, AGAL deposit number NM97/09514 dated 18 Aug. 1997, or a derivative thereof for use in inhibiting the growth of methane-producing bacteria and/or archaea in the forestomach of ruminant animals, reducing the ability of the rumen microbiome to produce methane, reducing methane emissions by a ruminant animal, increasing feed efficiency in a ruminant animal, increasing the yield of milk and/or milk components produced from a ruminant animal, or improving the body weight and/or body composition of a ruminant animal.

In a further aspect, the invention provides use of Lacticaseibacillus rhamnosus strain HN001, AGAL deposit number NM97/09514 dated 18 Aug. 1997, or a derivative thereof for inhibiting the growth of methane-producing bacteria and/or archaea in the forestomach of ruminant animals, reducing the ability of the rumen microbiome to produce methane, reducing ruminal methane production by a ruminant animal, increasing feed efficiency in a ruminant animal, increasing the yield of milk and/or milk components produced from a ruminant animal, or improving the body weight and/or body composition of a ruminant animal.

This invention may also be said broadly to consist in the parts, elements and features referred to or indicated in the specification of the application, individually or collectively, and any or all combinations of any two or more said parts, elements or features, and where specific integers are mentioned herein which have known equivalents in the art to which this invention relates, such known equivalents are deemed to be incorporated herein as if individually set forth.

It is intended that reference to a range of numbers disclosed herein (for example, 1 to 10) also incorporates reference to all rational numbers within that range (for example, 1, 1.1, 2, 3, 3.9, 4, 5, 6, 6.5, 7, 8, 9 and 10) and also any range of rational numbers within that range (for example, 2 to 8, 1.5 to 5.5 and 3.1 to 4.7) and, therefore, all sub-ranges of all ranges expressly disclosed herein are hereby expressly disclosed. These are only examples of what is specifically intended and all possible combinations of numerical values between the lowest value and the highest value enumerated are to be considered to be expressly stated in this application in a similar manner.

The term “comprising” as used in this specification means “consisting at least in part of”. When interpreting each statement in this specification that includes the term “comprising”, features other than that or those prefaced by the term may also be present. Related terms such as “comprise” and “comprises” are to be interpreted in the same manner.

In this specification where reference has been made to patent specifications, other external documents, or other sources of information, this is generally for the purpose of providing a context for discussing the features of the invention. Unless specifically stated otherwise, reference to such external documents is not to be construed as an admission that such documents, or such sources of information, in any jurisdiction, are prior art, or form part of the common general knowledge in the art.

### BRIEF DESCRIPTION OF THE DRAWINGS

FIG. 1. Impact of L. rhamnosus HN001 culture or supernatant (SN) on pH in the rumen in vitro experiment compared to control solution (Buffer).

FIG. 2. Proportions of the main VFAs measured in rumen in vitro fermentations inoculated with HN001™, SN, or Buffer.

FIG. 3. Minor VFAs in rumen in vitro samples inoculated with HN001™, SN, or Buffer.

FIG. 4. Proportions of the main VFAs produced during rumen fermentations (A: Acetic acid, B: Butyric acid, C: Propionic acid) inoculated with HN001™, SN, or Buffer. T-test HN001™ vs Buffer **p<0.01; ***p<0.001; SN vs Buffer p<0.05, ‡‡ p<0.01, ‡‡‡ p<0.001.

FIG. 5. Lactic acid concentration in rumen in vitro fermentations treated with HN001™, SN, or Buffer. T-test HN001™ vs Buffer **p<0.01; ***p<0.001; SN vs Buffer; ‡‡‡ p<0.001; HN001™ vs SN ### p<0.001.

FIG. 6. Relative abundance of bacterial phyla in rumen in vitro fermentations treated with HN001™, SN, or Buffer. T-test vs Buffer *p<0.05; **p<0.01. The stacked bars from bottom to top correspond to the phyla in the figure legend from left to right, i.e. the bottom bar is Firmicutes, the bar above that is Bacteroidetes, etc.

FIG. 7. Relative abundance of Lactobacillus strain identified in rumen in vitro assay treated with HN001™, SN, or Buffer. The stacked bars from bottom to top correspond to the strains in the figure legend from left to right.

FIG. 8. Diversity and relative abundance of Archaea in rumen in vitro fermentations treated with HN001™, SN or Buffer. The stacked bars from bottom to top correspond to the species in the figure legend from left to right.

FIG. 9. Diversity and relative abundance of protozoa species at 0, 6 and 48 hours in rumen in vitro assay treated with HN001™, SN or Buffer. The stacked bars from bottom to top correspond to the species in the figure legend from left to right.

### DETAILED DESCRIPTION OF THE INVENTION

The present invention is based on the finding that lactic acid bacteria strain Lacticaseibacillus rhamnosus strain HN001 (formerly classified as Lactobacillus rhamnosus HN001) and derivatives thereof inhibit or suppress the growth of methane-producing bacteria and/or archaea in the forestomach of ruminant animals and/or reduce the ability of the rumen microbiota to produce methane. Inhibiting the growth of methane-producing bacteria and/or archaea can reduce ruminal methane production and increase volatile fatty acids (VFAs) in the rumen and forestomach, which can act as an increased energy source driving enhanced growth or increased productivity, such as milk or meat production, and can stimulate rumen development, such as rumen papillae development.

Accordingly, in a first aspect the invention provides a method for inhibiting the growth of methane-producing bacteria and/or archaea in the forestomach of ruminant animals, or for reducing the ability of the rumen microbiome to produce methane, wherein the method comprises administering to a ruminant animal an effective amount of Lacticaseibacillus rhamnosus strain HN001, AGAL deposit number NM97/09514 dated 18 Aug. 1997, or a derivative thereof.

In a second aspect, the invention provides a method for reducing ruminal methane production by a ruminant animal, wherein the method comprises administering to the animal an effective amount of Lacticaseibacillus rhamnosus strain HN001, AGAL deposit number NM97/09514 dated 18 Aug. 1997, or a derivative thereof.

In a third aspect the invention provides a method for increasing feed efficiency in a ruminant animal, wherein the method comprises administering to the animal an effective amount of Lacticaseibacillus rhamnosus strain HN001, AGAL deposit number NM97/09514 dated 18 Aug. 1997, or a derivative thereof.

In a fourth aspect, the invention provides a method for improving the absorptive capacity of the forestomach, for example increasing the absorptive capacity for volatile fatty acids (VFAs), wherein the method comprises administering to the animal an effective amount of Lacticaseibacillus rhamnosus strain HN001, AGAL deposit number NM97/09514 dated 18 Aug. 1997, or a derivative thereof.

In a fifth aspect, the invention provides a method for enhancing the physical and/or functional development of the rumen in a young ruminant, for example a young ruminant prior to weaning, wherein the method comprises administering to the animal an effective amount of Lacticaseibacillus rhamnosus strain HN001, AGAL deposit number NM97/09514 dated 18 Aug. 1997, or a derivative thereof.

In one embodiment, the methods and compositions disclosed herein enhance anatomical development of the rumen. For example, the methods and compositions enhance development of rumen epithelium and/or muscularisation, for example increasing growth of rumen mass, growth of rumen papillae, increase in papillae density, for example dorsal papillae density, and/or total surface area of the ruminal wall in the animal.

In one embodiment, the methods and compositions disclosed herein enhance rumen weight, ruminal wall thickness, or density of rumen papillae per cm² of ruminal wall.

In one embodiment, the methods and compositions disclosed herein enhance functional achievement of the rumen. For example, the method stimulates rumination and/or enhances dry matter intake (DMI). In one embodiment the methods and compositions disclosed herein increase ruminal turnover rate and/or increase post-ruminal digestion. Without wishing to be bound by theory, it has been hypothesised that a higher rumen turnover rate selects for microorganisms that are capable of fast, heterofermentative growth on soluble sugars, producing less hydrogen, which leads to less methane formation.

The term “administering” refers to the action of introducing an effective amount of Lacticaseibacillus rhamnosus strain HN001 into the forestomach of a ruminant animal. More particularly, this administration is an administration by oral route. This administration can in particular be carried out by supplementing the feed or drink intended for the animal with the strain; the supplemented feed or drink then being ingested by the animal.

The term “effective amount” refers to a quantity of Lacticaseibacillus rhamnosus strain HN001 sufficient to allow a desired effect, i.e., inhibition of the growth of methane-producing bacteria and/or archaea in the forestomach of the animal, a reduction in methane production or emission by the animal, or an increase in feed efficiency in the animal, in comparison with a reference. The desired effect (such as inhibition of growth of methane-producing bacteria and/or archaea and/or reduction of methane production or emission) can be measured in vitro or in vivo. For example, the desired effect can be measured in vitro using the methods described herein, for example, in the Examples below, in an artificial rumen system, such as that described in T. Hano (1993) J. Gen. Appl. Microbial., 39, 35-45, or by in vivo oral administration to ruminants.

This effective amount can be administered to the ruminant animal in one or more doses.

The term “reducing methane production”, e.g., “reducing methane production by the animal” refers to reducing methane production by any mechanism, and from any ruminant-related source. For example, the term may refer to a reduction in methane produced within the forestomach of ruminant animals, or it may refer to a reduction in methane produced or emitted by the faeces of a ruminant animal.

It is anticipated that the reduction in methane production may be due to a variety of mechanisms. These may include, for example, killing methanogens (i.e. a bactericidal/archaeacidal effect), inhibiting the growth of methanogens (i.e. a bacteriostatic/archaeostatic effect), and/or inhibiting the ability of the forestomach or rumen microbiota to produce methane. Inhibiting the ability of the forestomach or rumen microbiota to produce methane may be via a variety of mechanisms, including, for example, physical and/or chemical changes to the forestomach or rumen environment, changes to the microbiota, the inhibition of one or more methanogenic pathways, and/or cross-feeding (or disrupting cross-feeding) of intermediaries between members of the microbiome.

The term “feed efficiency” refers to the ability of an animal to turn feed nutrients into milk or milk components, protein (such as muscle) and/or fat. Microbial fermentation in the forestomach or rumen produce volatile fatty acids (VFA) such as acetic acid, propionic acid and butyric acid. These fatty acids are absorbed directly from the rumen wall and used as raw materials for milk components and other final digested products, and the majority of the energy consumed by body tissues is used to produce milk or milk components, or muscle. Thus, when the utilisation of energy is improved, milk production, e.g., milk yield, and/or milkfat, milk protein, and/or milk solids can be increased. Increases in muscle, and/or improvements in body composition, such as altered muscle/fat ratio in an animal, can also be achieved.

Feed efficiency can be calculated by dividing the weight of milk produced by an animal by the weight of dry matter consumed by that animal. Thus, an animal with a higher feed efficiency will produce more milk, or milk with a higher content of milk components such as, but not limited to, fat and protein, than an animal with a lower feed efficiency when given the same nutrient input. Feed efficiency can be measured by differences in the growth of an animal by any of the following parameters: average daily weight gain, total weight gain, feed conversion, which includes both feed:gain and gain:feed, feed efficiency, mortality, and feed intake. The feed efficiency may be standardised to account for differences in protein and fat content by using the energy-corrected milk (ECM) yield instead of the weight of milk. This can be calculated using the following formula (Tyrrell and Reid, 1965):

ECM=(12.82×weight of fat in pounds)+(7.13×weight of protein in pounds)+(0.323×weight of milk in pounds).

In one embodiment, the feed efficiency in a ruminant animal is increased to at least about 1.01× of the feed efficiency of an untreated animal, such as at least about 1.02×, 1.03×, 1.04×, 1.05×, 1.06×, 1.07×, 1.08×, 1.09×, 1.10×, 1.12×, 1.14×, 1.16×, 1.18×, such as at least about 1.20×.

In some embodiments, L. rhamnosus HN001 or a derivative thereof promotes propionic acid production. Propionic acid has higher ATP production efficiency compared with other volatile fatty acids, and hence, feed efficiency is improved owing to the promotion of propionic acid production. Propionic acid is also glucogenic and can thus promote lactose synthesis in the mammary gland.

In some embodiments, L. rhamnosus HN001 or a derivative thereof shifts hydrogen metabolism from methanogenesis to short chain/volatile fatty acid (VFA) production, for example to propionic acid production. Propionate is predominantly used as a glucose precursor in ruminants, and more propionate formation would likely result in a more efficient utilisation of feed energy. Maximizing the flow of metabolic hydrogen in the forestomach or rumen away from methane and toward VFA (mainly propionate) would increase the efficiency of ruminant production and decrease its environmental impact, and would enhance rumen development and/or rumen papillae development.

Acetate is the primary substrate for mammary lipid synthesis, along with β-hydroxybutyrate which is produced during the absorption of butyrate. Consequently, a high acetate fermentation pattern will provide substrate to maintain or increase milk fat.

Thus, in some embodiments, L. rhamnosus HN001 or a derivative thereof results in an increase in milkfat, milk protein, overall milk volume and/or milk solids as a result of increased VFAs in the forestomach or rumen, which can act as an increased energy source driving increased production.

In some embodiments, the yield of milk and/or or milk components produced from the animal are preferably increased by 1.5% or more, more preferably, by 3.0% or more, by 4.5% or more, or by 6.0% or more.

It is anticipated that the present invention could also be used to extend the lactation cycle of a lactating ruminant, such as a cow. A cow directs a significant portion of its energy towards producing milk during lactation. After a long period of lactation, its body condition will be poorer for it. Because of this, the lactation period is usually shortened or curtailed to prevent excess deterioration on body condition. It is anticipated that the methods and ruminant feed composition disclosed herein will increase feed efficiency by the ruminant animal and therefor reduce the impact of milk production on body condition. As a result it would be possible to milk cows for a longer duration.

It is also anticipated that the present invention could also be used to reduce or ameliorate the deterioration of body condition due to lactation. It is anticipated that the methods and ruminant feed compositions disclosed herein will increase feed efficiency by the ruminant animal and therefore result in the ruminant animal having an improved body condition at the end of lactation. For example, the animal has a higher body condition score (BCS) when the animal enters the dry period. As a result, the ruminant animal would require less dry matter intake during the offseason to gain body condition. Alternatively or additionally, the methods and ruminant feed compositions disclosed herein are useful for improving body condition of an animal prior to lactation. For example, the methods and compositions disclosed herein could improve the body composition of the mother and/or the foetus or neonate. For example, the methods and compositions disclosed herein could improve body composition and/or weight of the neonate at birth.

It is also anticipated that the present invention could be similarly useful for reducing or ameliorating the deterioration of body condition in other times of stress, such as calving, drought, or insufficient feed intake.

As discussed above, the methods and compositions disclosed herein enhance the physical and/or functional development of the rumen, particularly in early life of young or pre-weaning ruminants. The development of the rumen involves three distinct processes: (i) anatomical development (e.g., growth in rumen mass and growth of rumen papillae), (ii) functional achievement (e.g., fermentation capacity and enzyme activity), and (iii) microbial colonization (bacteria, fungi, methanogens, and protozoa).

The anatomical development of the rumen is a process that occurs following three phases: non-rumination (0-3 weeks), transitional phase (3-8 weeks), and rumination (from 8 weeks on. During the transitional phase, growth and development of the ruminal absorptive surface area (papillae) is essential to enable absorption and utilisation of digestion end products, specifically rumen volatile fatty acids. The presence and absorption of volatile fatty acids stimulates rumen epithelial metabolism and may be key in initiating rumen epithelial development. A continuous exposure to volatile fatty acids maintains rumen papillae growth, size, and function. Different volatile fatty acids stimulate such development differently, with butyrate the most stimulatory, followed by propionate. Thus, it is expected that shifts hydrogen metabolism from methanogenesis to short chain/volatile fatty acid (VFA) production, for example to propionic acid production, would therefore enhance rumen epithelial growth and development.

### Ruminants

Ruminants are a group of herbivores having a stomach comprising multiple compartments, that digest their food by a first microbial fermentation in the rumen to form a cud, regurgitating and chewing the cud, and then swallowing the chewed cud for further digestion. This group includes, but is not limited to, the Ruminantia and Tylopoda suborders, and includes several species of domesticated livestock. In one embodiment, the ruminant animal is a bovine, goat, sheep, bison, yak, water buffalo, deer, camel, alpaca, llama, wildebeest, antelope, or nilgai. In a preferred embodiment, the ruminant animal is a bovine or a sheep.

In one embodiment, the ruminant animal is a lactating animal. In an alternative embodiment, the ruminant animal is a pre-weaning animal, such as a calf or a lamb.

The ruminant stomach is divided into the nonglandular forestomach (rumen, reticulum, omasum) and the terminal glandular stomach, the abomasum.

In some embodiments, the ruminant animal is neonatal, newborn, or young. For example, in some embodiments, the ruminant animal is one day, 2 days, 3 days, 4 days, 5 days, 6 days, 1 week, 2 weeks, 3 weeks, one month, or 2 months of age.

In some embodiments, the L. rhamnosus HN001 or derivative thereof is administered to the ruminant animal prior to weaning. In some embodiments, the L. rhamnosus HN001 or derivative thereof is administered to the ruminant animal after weaning. In some embodiments, the L. rhamnosus HN001 or derivative thereof is administered to the ruminant animal both prior to weaning and after weaning.

For example, the L. rhamnosus HN001 or derivative thereof is administered to the ruminant animal on or about day 0 of birth, for example around day 0, day 1 or day 2 of birth. Administration may then occur at least one per day, for example multiple times per day, sufficient to obtain persistency of effect. For example, administration may continue for 2 days, 3 days, 4 days, 5 days, 6 days, 1 week, 2 weeks, 3 weeks, one month, 6 weeks, 2 months, 10 weeks or three months from birth.

### Lacticaseibacillus rhamnosus HN001

As described in the applicant's PCT International application PCT/NZ98/00122 (published as WO 99/10476 and incorporated herein in its entirety), a freeze-dried culture of Lacticaseibacillus rhamnosus HN001 (formerly classified as Lactobacillus rhamnosus HN001) was deposited at the Australian Government Analytical Laboratories (AGAL), The New South Wales Regional Laboratory, 1 Suakin Street, Pymble, NSW 2073, Australia, on 18 Aug. 1997 and was accorded deposit number NM97/09514. This Budapest Treaty-recognised depository is now no longer referred to as AGAL, but rather is referred to as the National Measurement Institute of Australia (NMIA). The genome sequence of L. rhamnosus HN001 is available at Genbank under accession number: NZ_ABWJ00000000. The terms Lacticaseibacillus rhamnosus HN001, Lactobacillus rhamnosus HN001, L. rhamnosus HN001 and HN001™ are used interchangeably herein. HN001™ is a trade mark of Fonterra™ Limited.

### Morphological Properties

The morphological properties of L. rhamnosus HN001 are described below.

Short to medium rods with square ends in chains, generally 0.7×1.1×2.0−4.0 μm, when grown in MRS broth.

Gram positive, non-mobile, non-spore forming, catalase negative facultative anaerobic rods with optimum growth temperature of 37±1° C. and optimum pH of 6.0-6.5. These are facultatively heterofermentative bacteria and no gas is produced from glucose.

### Fermentative Properties

An API 50 CH sugar fermentation kit was used to determine the carbohydrate fermentation pattern of L. rhamnosus HN001, yielding a score of 5757177 (based on scores of 22 prominent sugars—see PCT/NZ98/00122).

### Further Characterisation

L. rhamnosus strain HN001 may be further characterised by the functional attributes disclosed in PCT/NZ98/00122, including its ability to adhere to human intestinal epithelial cells, and by the improvements in phagocyte function, in antibody responses, in natural killer cell activity, and in lymphocyte proliferation elicited by dietary intake or in in vitro model systems. It will be appreciated that there are a wide variety of methods known and available to the skilled artisan that can be used to confirm the identity of L. rhamnosus HN001, wherein exemplary methods include DNA fingerprinting, genomic analysis, sequencing, and related genomic and proteomic techniques.

### L. rhamnosus HN001 and Derivatives Thereof

As described herein, certain embodiments of the present invention utilise live L. rhamnosus HN001. In other embodiments, an L. rhamnosus HN001 derivative is utilised.

As used herein, the term “derivative” and grammatical equivalents thereof when used with reference to bacteria (including use with reference to a specific strain of bacteria such as L. rhamnosus HN001) contemplates mutants and homologues of or derived from the bacteria, killed or attenuated bacteria such as but not limited to heat-killed, lysed, fractionated, pressure-killed, irradiated, and UV- or light-treated bacteria, and material derived from the bacteria including but not limited to bacterial cell wall compositions, bacterial cell lysates, lyophilised bacteria, anti-methanogen factors from the bacteria, bacterial metabolites, bacterial cell suspensions, bacterial culture supernatant, and the like, wherein the derivative retains anti-methanogen activity. Transgenic microorganisms engineered to express one or more anti-methanogen factors are also contemplated. Methods to produce such derivatives, such as but not limited to one or more mutants of L. rhamnosus HN001 or one or more anti-methanogen factors, and particularly derivatives suitable for administration to a ruminant animal (for example, in a composition) are well known in the art.

It will be appreciated that methods suitable for identifying L. rhamnosus HN001, such as those described above, are similarly suitable for identifying derivatives of L. rhamnosus HN001, including for example mutants or homologues of L. rhamnosus HN001, or for example bacterial metabolites from L. rhamnosus HN001.

The term “anti-methanogen factor” refers to a bacterial molecule responsible for mediating anti-methanogen activity, including but not limited to bacterial DNA motifs, proteins, bacteriocins, bacteriocin-like molecules, anti-microbial peptides, antibiotics, antimicrobials, small molecules, polysaccharides, or cell wall components such as lipoteichoic acids and peptidoglycan, or a mixture of any two or more thereof. While, as noted above, these molecules have not been clearly identified, and without wishing to be bound by any theory, their presence can be inferred by the presence of anti-methanogen activity.

The term “anti-methanogen activity” refers to the ability of certain microorganisms to inhibit the growth of methanogenic bacteria and/or archaea, and/or to reduce the production of methane by methanogenic bacteria and/or archaea. This ability may be limited to inhibiting the growth of and/or ability to produce methane of certain groups of methanogenic bacteria and/or archaea such as, for example, inhibiting the growth of hydrogenotrophic methanogens, inhibiting the ability of hydrogenotrophic methanogens to produce methane, inhibiting the growth of methylotrophic methanogens, inhibiting the ability of methylotrophic methanogens to produce methane, inhibiting the growth of certain species of methanogens, or inhibiting the ability of certain species of methanogens to produce methane.

Reference to retaining anti-methanogen activity is intended to mean that a derivative of a microorganism, such as a mutant or homologue of a microorganism or an attenuated or killed microorganism, or a cell culture supernatant, still has useful anti-methanogen activity, or that a composition comprising a microorganism or a derivative thereof still has useful anti-methanogen activity. While the bacterial molecules responsible for mediating anti-methanogen activity have not been clearly identified, molecules that have been proposed as possible candidates include bacterial DNA motifs, proteins, bacteriocins, antibiotics, surface proteins, small organic acids, polysaccharides, and cell wall components such as lipoteichoic acids and peptidoglycan. It has been postulated that these interact with components of the methanogenic bacteria and/or archaea to give a growth-inhibitory effect. Preferably, the retained activity is at least about 35, 40, 45, 50, 55, 60, 65, 70, 75, 80, 85, 90, 95, 99 or 100% of the activity of an untreated (i.e., live or non-attenuated) control, and useful ranges may be selected between any of these values (for example, from about 35 to about 100%, from about 50 to about 100%, from about 60 to about 100%, from about 70 to about 100%, from about 80 to about 100%, and from about 90 to about 100%).

Using conventional solid substrate and liquid fermentation technologies well known in the art, L. rhamnosus HN001 can be grown in sufficient amounts to allow use as contemplated herein. For example, L. rhamnosus HN001 can be produced in bulk for formulation using nutrient film or submerged culture growing techniques, for example under conditions as described in WO99/10476. Briefly, growth is carried out under aerobic conditions at any temperature satisfactory for growth of the organism. For example, for L. rhamnosus HN001 a temperature range of from 30 to 40° C., preferably 37° C., is preferred. The pH of the growth medium is slightly acidic, preferably about 6.0 to 6.5. Incubation time is sufficient for the isolate to reach a stationary growth phase.

L. rhamnosus HN001 cells may be harvested by methods well known in the art, for example, by conventional filtering or sedimentary methodologies (e.g. centrifugation) or harvested dry using a cyclone system. L. rhamnosus HN001 cells can be used immediately or stored, preferably freeze-dried or chilled at −20° to 6° C., preferably −4° C., for as long as required using standard techniques.

### Supernatants

Further embodiments of the present invention utilise supernatant(s) from a cell culture comprising L. rhamnosus HN001 or a derivative thereof. These embodiments include processes for preparing an L. rhamnosus HN001 supernatant, said process comprising culturing cells of L. rhamnosus HN001, and separating the supernatant from the cultured cells, thereby obtaining the supernatant. This process also enables further isolation of bacterial molecules responsible for mediating anti-methanogen activity that are obtainable from the supernatant.

As would be understood by the skilled addressee, a supernatant useful in the present invention encompasses both the supernatant from such cultures, and/or concentrates of such supernatant and/or fractions of such supernatant.

The term “supernatant” in the present context refers to a medium from a bacterial culture from which the bacteria have subsequently been removed, e.g. by centrifugation or filtration.

A supernatant useful in the present invention can readily be obtained by a simple process for preparing an L. rhamnosus HN001 supernatant, said process comprising
a) culturing cells of L. rhamnosus HN001, and
b) optionally releasing of active compounds and/or extracellular components of the cells by various cellular treatments such as, but not limited to, acidic or alkaline modifications, sonication, detergents e.g. Sodium dodecyl sulfate (SDS) and/or Triton X, muralytic enzymes e.g. mutalolysin and/or lysozyme, salt and/or alcohol;
c) separating the supernatant from the cultured cells,

thereby obtaining said supernatant.

In a preferred embodiment of this process, the supernatant composition is further subjected to a drying step to obtain a dried culture product.

The drying step may conveniently be freeze drying or spray drying, but any drying process which is suitable for drying of anti-methanogen factors such as bacteriocins, also including vacuum drying and air drying, are contemplated.

Although the content of the supernatant produced by L. rhamnosus HN001 is not yet characterised in detail, it is known that certain Lactobacillus may produce bacteriocins that are small heat-stable proteins and therefore, without wishing to be bound by theory, it is expected that even drying methods, including spray drying, which result in moderate heating of the culture eluate product, will result in active compositions, as demonstrated in the Examples described herein.

### Lysate

A fluid containing the contents of lysed cells is called a lysate. A lysate contains active components of the bacterial cells and may be either crude, thus containing all cellular components, or partially and/or completely separated in separate fractions, such as extracellular components, intracellular components, proteins etc.

Methods for producing bacterial cell lysates are well known in the art. Such methods can include, but are not limited to, mechanical lysis, such as mechanical shearing, grinding, milling, or sonication, enzymatic lysis, such as by enzymes that degrade the bacterial cell wall, chemical lysis, such as using detergents, denaturants, pressure alterations, and/or osmotic shock, and combinations of the above.

Further embodiments of the present invention thus utilise a lysate of L. rhamnosus HN001 or a derivative thereof.

### Cell Suspension

The present invention may also in some embodiments utilise a cell suspension comprising L. rhamnosus HN001 or a derivative thereof.

In the present context, the term “cell suspension” relates to a number of L. rhamnosus HN001 or a derivative thereof dispersed or in suspension in a liquid e.g. a liquid nutrient medium, culture medium or saline solution.

The cells may be presented in the form of a cell suspension in a solution that is suitable for dispersion. The cell suspension can e.g. be dispersed via spraying, dipping, or any other application process.

The cells may be viable, but the suspension may also comprise inactivated or killed cells or a lysate hereof. In one embodiment, the suspension of the present invention comprises viable cells. In another embodiment, the suspension of the present invention comprises inactivated, killed or lysed cells.

### Bacteriocins

Bacteriocins are antimicrobial compounds produced by bacteria to inhibit other bacterial strains and species.

Lactic acid bacteria (LAB) are well known to produce bacteriocins and these compounds are of global interest to the food industry because they inhibit the growth of many spoilage and pathogenic bacteria, thus extending shelf life and safety of foods. Bacteriocins are typically considered to be narrow spectrum antibiotics. Moreover, bacteriocins of especially LAB display very low human toxicity and have been consumed in fermented food for millennia.

As is illustrated in the Examples disclosed herein, it has been found that L. rhamnosus HN001, or compositions comprising L. rhamnosus HN001, and the culture supernatant of L. rhamnosus HN001 are useful as an antimicrobial compound, in particular for inhibiting the growth of methane-producing bacteria and/or inhibiting the ability of methanogens to produce methane.

In the present context, the term antimicrobial compound utilises a compound that kills microorganisms, impair their survival or inhibits their growth.

Antimicrobial compounds can be grouped according to the microorganisms they act primarily against. For example, antibacterials are used against bacteria and antifungals are used against fungi. They can also be classified according to their function. Compounds that kill microbes are called microbicidal, while those that merely inhibit their growth are called microbiostatic.

In one embodiment, the present invention relates to an antimicrobial compound, which is microbicidal. In another embodiment, the present invention relates to an antimicrobial compound, which is microbiostatic. In another embodiment, the present invention relates to an antimicrobial compound, which is antibacterial.

### Ruminant Feed or Carrier Compositions

A ruminant feed composition useful herein may be formulated as a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or intra-ruminal product, e.g., a bolus. Appropriate formulations may be prepared by an art skilled worker with regard to that skill and the teaching of this specification.

The composition can be administered as a top dressing on, or mixed into, a standard feed material such as a daily ration. In addition, the strain can be administered in a partial or total mixed ration (TMR), pelleted feedstuff, mixed in with liquid feed or drink, mixed in a protein premix, or delivered via a vitamin and mineral premix.

In one embodiment, compositions useful herein include any edible feed product which is able to carry bacteria or a bacterial derivative. As used in this application, the term “feed(s)” or “animal feed(s)” refers to material(s) that are consumed by animals and contribute energy and/or nutrients to an animal's diet. Animal feeds typically include a number of different components that may be present in forms such as concentrate(s), premix(es), co-product(s), or pellets. Examples of feeds and feed components include Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, and molasses.

Other compositions useful as a carrier include milk, milk powder, milk replacement, milk fortifier, whey, whey powder, sucrose, maltodextrin, rice hulls.

In certain embodiments, the feed composition is formed through a process of growing L. rhamnosus HN001 using a milk-based carrier, such as thermalized milk, or a non-milk-based carrier, to create a fermented yoghurt-style composition. Methods to create such fermented yoghurt-style compositions are well known in the art, and may include, for example, using a warm water bath or other heating means to incubate the milk at a suitable temperature until a sufficient cell density is reached, such as over 12 hours. In one embodiment, the temperature is 25-30° C. Optionally, the milk may include other additives to promote bacterial growth, such as yeast extract. In certain embodiments, this method takes place on-site, such as on the farm where the probiotic feed supplementation is to take place. The fermented yoghurt-style composition may be administered by oral application, such as by drenching. In some embodiments, the fermented yoghurt-style composition is administered at a dose of 1-100 ml per day, such as 2-50, 5-30, or 10-20 ml per day.

Other suitable feed formulations for ruminants are described in E. W. Crampton et al., Applied Animal Nutrition, W. H. Freeman and Company, San Francisco, CA., 1969 and D. C. Church, Livestock Feeds and Feeding, O & B Books, Corvallis, Oreg., 1977, both of which are incorporated herein by reference.

In one embodiment, compositions useful herein include any non-feed carrier consumed by the animal to which bacteria or a bacterial derivative is added, such as vermiculite, zeolites or crushed limestone and the like.

In certain embodiments, the composition of the invention comprises live L. rhamnosus HN001. Methods to produce such compositions are well known in the art.

In some embodiments, the composition of the invention comprises one or more L. rhamnosus HN001 derivatives. Again, methods to produce such compositions are well known in the art and may utilise standard microbiological and pharmaceutical practices. In some embodiments, the composition comprises a dried culture product, such as a supernatant or cell lysate as described herein.

It will be appreciated that a broad range of additives or carriers may be included in such compositions, for example to improve or preserve bacterial viability or to increase anti-methanogen activity of L. rhamnosus HN001 or of one or more L. rhamnosus HN001 derivatives. For example, additives such as surfactants, wetters, humectants, stickers, dispersal agents, stabilisers, penetrants, and so-called stressing additives to improve bacterial cell vigour, growth, replication and survivability (such as potassium chloride, glycerol, sodium chloride and glucose), as well as cryoprotectants such as maltodextrin, may be included. Additives may also include compositions which assist in maintaining microorganism viability in long term storage, for example unrefined corn oil, or “invert” emulsions containing a mixture of oils and waxes on the outside and water, sodium alginate and bacteria on the inside.

In some embodiments, the L. rhamnosus HN001 or derivative thereof are encapsulated. Methods to produce such encapsulated bacteria are well known in the art. In some embodiments, the L. rhamnosus HN001 or derivative thereof are encapsulated in liposomes, microbubbles, microparticles or microcapsules and the like. Such encapsulants can include natural, semisynthetic, or synthetic polymers, waxes, lipids, fats, fatty alcohols, fatty acids, and/or plasticisers, for example alginates, gums, κ-Carrageenan, chitosan, starch, sugars, gelatine, and so on.

In certain embodiments, the L. rhamnosus HN001 is in a reproductively viable form and amount.

The composition may comprise a carbohydrate source, such as a disaccharide including, for example, sucrose, fructose, glucose, or dextrose. Preferably the carbohydrate source is one able to be aerobically or anaerobically utilised by L. rhamnosus HN001.

In such embodiments, the composition preferably is capable of supporting reproductive viability of the L. rhamnosus HN001 for a period greater than about two weeks, preferably greater than about one month, about two months, about three months, about four months, about five months, more preferably greater than about six months, most preferably at least about 2 years to about 3 years or more.

In certain embodiments, an oral composition is formulated to allow the administration of an effective amount of L. rhamnosus HN001 to establish a population in the gastrointestinal tract of the animal when ingested. The established population may be a transient or permanent population.

While various routes and methods of administration are contemplated, oral administration of L. rhamnosus HN001, such as in a composition suitable for oral administration, is currently preferred. It will of course be appreciated that other routes and methods of administration may be utilised or preferred in certain circumstances.

The term “oral administration” includes oral, buccal, enteral, intra-ruminal, and intra-gastric administration.

In theory one colony forming unit (cfu) should be sufficient to establish a population of L. rhamnosus HN001 in an animal, but in actual situations a minimum number of units are required to do so. Therefore, for therapeutic mechanisms that are reliant on a viable, living population of probiotic bacteria, the number of units administered to a subject will affect efficacy.

In one embodiment, a composition formulated for administration will be sufficient to provide at least about 6×10⁹ cfu L. rhamnosus HN001 per day, for example at least about 6×10¹¹ cfu L. rhamnosus HN001 per day. In another embodiment, a composition formulated for administration will be sufficient to provide at least about 10¹² cfu L. rhamnosus HN001 per day.

Methods to determine the presence of a population of gut and/or rumen flora, such as L. rhamnosus HN001, in the gastrointestinal tract of a subject are well known in the art, and examples of such methods are presented herein. In certain embodiments, presence of a population of L. rhamnosus HN001 can be determined directly, for example by analysing one or more samples obtained from an animal and determining the presence or amount of L. rhamnosus HN001 in said sample. In other embodiments, presence of a population of L. rhamnosus HN001 can be determined indirectly, for example by observing a reduction in methane emissions or methane production, a reduction in hydrogen production, or a decrease in the number of other gut and/or rumen flora in a sample obtained from an animal. Combinations of such methods are also envisaged.

The efficacy of a composition useful according to the invention can be evaluated both in vitro and in vivo. See, for example, the examples below. Briefly, the composition can be tested for its ability to inhibit the growth of methanogenic bacteria and/or archaea, or its ability to reduce the production of methane by methanogenic bacteria and/or archaea. For in vivo studies, the composition can be fed to or injected into a ruminant and its effects on ruminal methanogenic bacteria and/or archaea, and its effect on methane production are then assessed. Based on the results, an appropriate dosage range and administration route can be determined.

Methods of calculating appropriate dose may depend on the nature of the active agent in the composition. For example, when the composition comprises live L. rhamnosus HN001, the dose may be calculated with reference to the number of live bacteria present. For example, as described herein the examples the dose may be established by reference to the number of colony forming units (cfu) to be administered per day, or by reference to the number of cfu per kilogram dry feed weight.

By way of general example, the administration of from about 1×10⁶ cfu to about 1×10¹² cfu of L. rhamnosus HN001 per kg dry feed weight per day, preferably about 1×10⁶ cfu to about 1×10¹¹ cfu/kg/day, about 1×10⁶ cfu to about 1×10¹⁰ cfu/kg/day, about 1×10⁶ cfu to about 1×10⁹ cfu/kg/day, about 1×10⁶ cfu to about 1×10⁸ cfu/kg/day, about 1×10⁶ cfu to about 5×10⁷ cfu/kg/day, or about about 1×10⁶ cfu to about 1×10⁷ cfu/kg/day, is contemplated. Preferably, the administration of from about 5×10⁶ cfu to about 5×10⁸ cfu per kg dry feed weight of L. rhamnosus HN001 per day, preferably about 5×10⁶ cfu to about 4×10⁸ cfu/kg/day, about 5×10⁶ cfu to about 3×10⁸ cfu/kg/day, about 5×10⁶ cfu to about 2×10⁸ cfu/kg/day, about 5×10⁶ cfu to about 1×10⁸ cfu/kg/day, about 5×10⁶ cfu to about 9×10⁷ cfu/kg/day, about 5×10⁶ cfu to about 8×10⁷ cfu/kg/day, about 5×10⁶ cfu to about 7×10⁷ cfu/kg/day, about 5×10⁶ cfu to about 6×10⁷ cfu/kg/day, about 5×10⁶ cfu to about 5×10⁷ cfu/kg/day, about 5×10⁶ cfu to about 4×10⁷ cfu/kg/day, about 5×10⁶ cfu to about 3×10⁷ cfu/kg/day, about 5×10⁶ cfu to about 2×10⁷ cfu/kg/day, or about 5×10⁶ cfu to about 1×10⁷ cfu/kg/day, is contemplated.

In certain embodiments, periodic dose need not vary with body weight, dry feed weight or other characteristics of the subject. In such examples, the administration of from about 1×10⁶ cfu to about 1×10¹³ cfu of L. rhamnosus HN001 per day, preferably about 1×10⁶ cfu to about 1×10¹² cfu/day, about 1×10⁶ cfu to about 1×10¹¹ cfu/day, about 1×10⁶ cfu to about 1×10¹⁰ cfu/day, about 1×10⁶ cfu to about 1×10⁹ cfu/day, about 1×10⁶ cfu to about 1×10⁸ cfu/day, about 1×10⁶ cfu to about 5×10⁷ cfu/day, or about about 1×10⁶ cfu to about 1×10⁷ cfu/day, is contemplated.

In certain embodiments, the administration of from about 5×10⁷ cfu to about 5×10¹⁰ cfu per kg body weight of L. rhamnosus HN001 per day, preferably about 5×10⁷ cfu to about 4×10¹⁰ cfu/day, about 5×10⁷ cfu to about 3×10¹⁰ cfu/day, about 5×10⁷ cfu to about 2×10¹⁰ cfu/day, about 5×10⁷ cfu to about 1×10¹⁰ cfu/day, about 5×10⁷ cfu to about 9×10⁹ cfu/day, about 5×10⁷ cfu to about 8×10⁹ cfu/day, about 5×10⁷ cfu to about 7×10⁹ cfu/day, about 5×10⁷ cfu to about 6×10⁹ cfu/day, about 5×10⁷ cfu to about 5×10⁹ cfu/day, about 5×10⁷ cfu to about 4×10⁹ cfu/day, about 5×10⁷ cfu to about 3×10⁹ cfu/day, about 5×10⁷ cfu to about 2×10⁹ cfu/day, or about 5×10⁷ cfu to about 1×10⁹ cfu/day, is contemplated. Preferably, a dose of between 1×10⁸ and 1×10⁹ cfu/kg body weight per day is administered.

It will be appreciated that, in certain embodiments, the dose need not be administered daily. For example, the composition may be formulated to be administered every two days, twice weekly, weekly, fortnightly, or monthly. Alternatively, in certain embodiments, the composition may be formulated to be administered 2, 3, 4, 5, 6, 7, 8, 9, 10, 11, 12, 13, 14, 15, 16, 17, 18, 19, 20, 21, 22, 23, or 24 times per day, with every feed, or with every mouthful.

It will be appreciated that the composition is preferably formulated so as to allow the administration of an efficacious dose of L. rhamnosus HN001 or one or more derivatives thereof. The dose of the composition administered, the period of administration, and the general administration regime may differ between animals depending on such variables as mode of administration chosen, and the age, sex, body weight, and species of an animal. Furthermore, as described above the appropriate dose may depend on the nature of the active agent in the composition and the manner of formulation.

Furthermore, the dose of the composition may vary over time. For example, in some embodiments, an initial dosing regimen may be followed by a maintenance dosing regimen. It will be appreciated that a higher dose may be required to establish a population of L. rhamnosus HN001 in the animal, and a lower dose may be sufficient to maintain said population. Accordingly, in some embodiments, the initial dosing regimen comprises administering a higher dose and/or a more frequent dose than the maintenance dosing regimen. Preferably, the initial dosing regimen is efficacious to establish a population of L. rhamnosus HN001 in the animal, and preferably the maintenance dosing regimen is efficacious to maintain a population of L. rhamnosus HN001 in the animal. In some embodiments, the maintenance dosing regimen comprises administering a dose every day, every second day, twice weekly, weekly, fortnightly, or monthly.

In some embodiments, the effect of the methods described herein persist after the administration of the L. rhamnosus HN001. Without wishing to be bound by theory, it is anticipated that administration of L. rhamnosus HN001 as described herein may result in a long-lasting or even permanent changes in the forestomach or rumen of the ruminant animal. In some embodiments, the effect persists for 2 days after the last administration of L. rhamnosus HN001, such as 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus HN001. In a preferred embodiment, the effect persists for the life of the animal.

In examples where the composition comprises one or more L. rhamnosus HN001 derivatives, the dose may be calculated by reference to the amount or concentration of L. rhamnosus HN001 derivative to be administered per day. For example, when the bacteria are inactivated, the quantities described previously are calculated before inactivation. For a composition comprising L. rhamnosus HN001 culture supernatant, the dose may be calculated by reference to the concentration of L. rhamnosus HN001 culture supernatant present in the composition. The concentration of L. rhamnosus HN001 culture supernatant present in the composition may be calculated, for example, on the basis of the cfu of the culture. For example, a dosage of culture supernatant equivalent to 1×10⁹ cfu/day can be calculated from the total yield of the culture and the total volume of the culture supernatant.

It will be appreciated that preferred compositions are formulated to provide an efficacious dose in a convenient form and amount. In certain embodiments, such as but not limited to those where periodic dose need not vary with body weight or other characteristics of the animal, the composition may be formulated for unit dosage. It should be appreciated that administration may include a single daily dose or administration of a number of discrete divided doses as may be appropriate. For example, an efficacious dose of L. rhamnosus HN001 may be formulated into a feed for oral administration.

However, by way of general example, the inventors contemplate administration of from about 1 mg to about 1000 mg of a composition useful herein per day, preferably about 50 to about 500 mg per day, alternatively about 150 to about 410 mg/day or about 110 to about 310 mg/day. In one embodiment, the inventors contemplate administration of from about 0.05 mg to about 250 mg per kg body weight of a composition useful herein.

In one embodiment a composition useful herein comprises, consists essentially of, or consists of at least about 0.1, 0.2, 0.5, 1, 5, 10, 15, 20, 25, 30, 35, 40, 45, 50, 55, 60, 65, 70, 75, 80, 85, 90, 95, 99, 99.5, 99.8 or 99.9% by weight of L. rhamnosus HN001 or a derivative thereof and useful ranges may be selected between any of these foregoing values (for example, from about 0.1 to about 50%, from about 0.2 to about 50%, from about 0.5 to about 50%, from about 1 to about 50%, from about 5 to about 50%, from about 10 to about 50%, from about 15 to about 50%, from about 20 to about 50%, from about 25 to about 50%, from about 30 to about 50%, from about 35 to about 50%, from about 40 to about 50%, from about 45 to about 50%, from about 0.1 to about 60%, from about 0.2 to about 60%, from about 0.5 to about 60%, from about 1 to about 60%, from about 5 to about 60%, from about 10 to about 60%, from about 15 to about 60%, from about 20 to about 60%, from about 25 to about 60%, from about 30 to about 60%, from about 35 to about 60%, from about 40 to about 60%, from about 45 to about 60%, from about 0.1 to about 70%, from about 0.2 to about 70%, from about 0.5 to about 70%, from about 1 to about 70%, from about 5 to about 70%, from about 10 to about 70%, from about 15 to about 70%, from about 20 to about 70%, from about 25 to about 70%, from about 30 to about 70%, from about 35 to about 70%, from about 40 to about 70%, from about 45 to about 70%, from about 0.1 to about 80%, from about 0.2 to about 80%, from about 0.5 to about 80%, from about 1 to about 80%, from about 5 to about 80%, from about 10 to about 80%, from about 15 to about 80%, from about 20 to about 80%, from about 25 to about 80%, from about 30 to about 80%, from about 35 to about 80%, from about 40 to about 80%, from about 45 to about 80%, from about 0.1 to about 90%, from about 0.2 to about 90%, from about 0.5 to about 90%, from about 1 to about 90%, from about 5 to about 90%, from about 10 to about 90%, from about 15 to about 90%, from about 20 to about 90%, from about 25 to about 90%, from about 30 to about 90%, from about 35 to about 90%, from about 40 to about 90%, from about 45 to about 90%, from about 0.1 to about 99%, from about 0.2 to about 99%, from about 0.5 to about 99%, from about 1 to about 99%, from about 5 to about 99%, from about 10 to about 99%, from about 15 to about 99%, from about 20 to about 99%, from about 25 to about 99%, from about 30 to about 99%, from about 35 to about 99%, from about 40 to about 99%, and from about 45 to about 99%).

In one embodiment a composition useful herein comprises, consists essentially of, or consists of at least about 0.001, 0.01, 0.05, 0.1, 0.15, 0.2, 0.3, 0.4, 0.5, 1, 2, 3, 4, 5, 6, 7, 8, 9, 10, 11, 12, 13, 14, 15, 16, 17, 18 or 19 grams of L. rhamnosus HN001 or a derivative thereof and useful ranges may be selected between any of these foregoing values (for example, from about 0.01 to about 1 grams, about 0.01 to about 10 grams, about 0.01 to about 19 grams, from about 0.1 to about 1 grams, about 0.1 to about 10 grams, about 0.1 to about 19 grams, from about 1 to about 5 grams, about 1 to about 10 grams, about 1 to about 19 grams, about 5 to about 10 grams, and about 5 to about 19 grams).

In certain embodiments, a composition useful herein comprises, consists essentially of, or consists of at least about 10⁴, 10⁵, 10⁶, 10⁷, 10⁸, 10⁹, 10¹⁰, 10¹¹, 10¹², or 10¹³ colony forming units (cfu) of L. rhamnosus HN001 per kg dry weight of the composition, and useful ranges may be selected between any of these foregoing values (for example, from about 10⁵ to about 10¹³ cfu, from about 10⁶ to about 10¹² cfu, from about 10⁷ to about 10¹² cfu, from about 10⁸ to about 10¹¹ cfu, from about 10⁸ to about 10¹⁰ cfu, and from about 10⁸ to about 10⁹ cfu).

It will be apparent that the concentration of L. rhamnosus HN001 or one or more derivatives thereof in a composition formulated for administration may be less than that in a composition formulated for, for example, distribution or storage, and that the concentration of a composition formulated for storage and subsequent formulation into a composition suitable for administration must be adequate to allow said composition for administration to also be sufficiently concentrated so as to be able to be administered at an efficacious dose.

The compositions useful herein may be used alone or in combination with one or more other therapeutic agents. The therapeutic agent may be a food, drink, food additive, drink additive, food component, drink component, dietary supplement, vitamin or mineral premix, oil, oil blend, oil rich feed supplement, nutritional product, medical food, nutraceutical, medicament or pharmaceutical. The therapeutic agent may be a probiotic agent or a probiotic factor, and is preferably effective to inhibit the growth of methanogenic bacteria and/or archaea, or to reduce methane production by methanogenic bacteria and/or archaea. In some embodiments, the oil, oil blend, or oil rich feed supplement is palm kernel expeller (PKE) and/or PROLIQ.

When used in combination with another therapeutic agent, the administration of a composition useful herein and the other therapeutic agent may be simultaneous or sequential. Simultaneous administration includes the administration of a single dosage form that comprises all components or the administration of separate dosage forms at substantially the same time. Sequential administration includes administration according to different schedules, preferably so that there is an overlap in the periods during which the composition useful herein and other therapeutic agent are provided. Examples of other therapeutic agents include at least one microorganism of a different species or strain, a vaccine that inhibits methanogens or methanogenesis, and/or a natural or chemically-synthesised inhibitor of methanogenesis and/or methanogen inhibitor, such as bromoform.

Suitable agents with which the compositions useful herein can be separately, simultaneously or sequentially administered include one or more prebiotic agents, one or more probiotic agents, one or more postbiotic agents, one or more phospholipids, one or more gangliosides, other suitable agents known in the art, and combinations thereof.

Typically, the term prebiotic refers to a material that stimulates the growth and/or activity of bacteria in the animals' digestive system that have biologic activity. Prebiotics may be selectively fermented ingredients that allow specific changes, both in the composition and/or activity of the gastrointestinal microflora, which confer health benefits upon the host. Probiotics generally refer to microorganisms that contribute to intestinal microbial balance which in turn play a role in maintaining health, or providing other biologic activity. Many species of lactic acid bacteria (LAB) such as, Lactobacillus and Bifidobacterium are generally considered as probiotics, but some species of Bacillus, and some yeasts have also been found as suitable candidates. Postbiotics refer to non-viable bacterial products or metabolic byproducts from microorganisms such as probiotics, that have biologic activity in the host.

Useful prebiotics include galactooligosaccharides (GOS), short chain GOS, long chain GOS, fructooligosaccharides (FOS), short chain FOS, long chain FOS, inulin, galactans, fructans, lactulose, and any mixture of any two or more thereof. Some prebiotics are reviewed by Boehm G and Moro G (Structural and Functional Aspects of Prebiotics Used in Infant Nutrition, J. Nutr. (2008) 138(9):1818S-1828S), incorporated herein by reference. Other useful agents may include dietary fibre such as a fully or partially insoluble or indigestible dietary fibre.

Accordingly, in one embodiment L. rhamnosus HN001 or derivative thereof may be administered separately, simultaneously or sequentially with one or more agents selected from one or more probiotics, one or more prebiotics, one or more sources of dietary fibre, one or more galactooligosaccharides, one or more short chain galactooligosaccharides, one or more long chain galactooligosaccharides, one or more fructooligosaccharides, one or more short chain galactooligosaccharides, one or more long chain galactooligosaccharides, inulin, one or more galactans, one or more fructans, lactulose, or any mixture of any two or more thereof.

In certain embodiments, the composition comprises L. rhamnosus HN001 and one or more prebiotics, one or more probiotics, one or more postbiotics, one or more sources of dietary fibre. In certain embodiments, the prebiotic comprises one or more fructooligosaccharides, one or more galactooligosaccharides, inulin, one or more galactans, one or more fructans, lactulose, or any mixture of any two or more thereof.

Without wishing to be bound by theory, it is believed that co-culture and/or co-administration of two or more strains of lactic acid bacteria, such as three strains of lactic acid bacteria, can reduce the incidence of culture failure due to infection by bacteriophages. Accordingly, in certain embodiments, the composition comprises L. rhamnosus HN001 and one or more other strain of lactic acid bacteria, preferably two or more other strains of lactic acid bacteria. In other embodiments, the composition comprising L. rhamnosus HN001 is administered simultaneously or sequentially with one or more other compositions comprising one or more other strains of lactic acid bacteria, preferably two or more other strains of lactic acid bacteria.

It will be appreciated that different compositions of the invention may be formulated with a view to administration to a particular ruminant subject group. For example, the formulation of a composition suitable to be administered to cattle may differ to that suitable to be administered to a different ruminant, such as sheep. It should also be appreciated that compositions of the invention may be formulated differently to be suitable to be administered to ruminant animals of different ages. For example, the formulation of a composition suitable to be administered to calves or lambs may differ to that suitable to be administered to adult cows or sheep. In certain embodiments, a first composition may be formulated for administration to young animals, such as pre-weaning animals, in an initial dosing regimen, and a second composition may be formulated for administration to the same animals in a maintenance dosing regimen. In some embodiments, the first composition is formulated for pre-weaning animals and the second composition is formulated for post-weaning animals.

### Preparation of L. rhamnosus HN001

Direct-fed microbials (DFMs) and their use in methods to modulate ruminal function and improve ruminant performance is known in the art, as are methods for their production.

Briefly, L. rhamnosus HN001 can be cultured using conventional liquid or solid fermentation techniques. In at least one embodiment, the strain is grown in a liquid nutrient broth, to a level at which the highest number of spores are formed. The strain is produced by fermenting the bacterial strain, which can be started by scaling-up a seed culture. This involves repeatedly and aseptically transferring the culture to a larger and larger volume to serve as the inoculum for the fermentation, which can be carried out in large strainless steel fermenters in medium containing proteins, carbohydrates, and minerals necessary for optimal growth. Non-limiting exemplary media are MRS or TSB. However, other media can also be used. After the inoculum is added to the fermentation vessel, the temperature and agitation are controlled to allow maximum growth. Once the culture reaches a maximum population density, the culture is harvested by separating the cells from the fermentation medium. This is commonly done by centrifugation.

In one embodiment, to prepare the L. rhamnosus HN001 strain, the strain is fermented to a 1×10⁸ CFU/ml to about 1×10⁹ CFU/ml level. The bacteria are harvested by centrifugation, and the supernatant is removed. The pelleted bacteria can then be used to produce a DFM. In at least some embodiments, the pelleted bacteria are freeze-dried and then used to form a DFM. However, it is not necessary to freeze-dry the strain before using them. The strain can also be used with or without preservatives, and in concentrated, unconcentrated, or diluted form.

The count of the culture can then be determined. CFU or colony forming unit is the viable cell count of a sample resulting from standard microbiological plating methods. The term is derived from the fact that a single cell when plated on appropriate medium will grow and become a viable colony in the agar medium.

Since multiple cells may give rise to one visible colony, the term colony forming unit is a more useful unit measurement than cell number.

### EXAMPLES

### 1. Example 1—Plate-Based Screen of Lacticaseibacillus rhamnosus HN001 Against an Indicator Methanogen Strain

### 1.1 Materials and Methods

### 1.1.1 Methanogen Culture

The inoculum of an indicator methanogen strain (Methanobrevibacter boviskoreani JH1) for seeding the plate assay was grown in 9 mL BY medium (Joblin, 2005) supplemented with 0.2 mL of 3 M sodium formate, 0.2 mL of 10 M ethanol, 0.1 mL of Vitamin Solution (Janssen et al., 1997) and 0.1 mL of Coenzyme M Solution (Sigma Aldrich, 0.1 M) by syringe using anaerobic techniques. The head spaces of the tubes were pumped with pressurized O₂-free CO₂ to 180 kPa and the tubes were incubated at 39° C. without shaking until visible turbidity appeared after 3 to 5 days. Methane produced by the methanogen strain was measured by removing a sample of the headspace gases by syringe and injecting on to a gas chromatograph (GC; Aerograph Corporation, USA) equipped with a Thermal Conductivity Detector (TCD) and using nitrogen as the carrier gas. The gas inside the tubes was released by venting with a sterile needle to prevent over pressurization. The cultures were routinely observed via wet mounts under fluorescence microscopy, and the methanogen strain appears as short ovoid-shaped rods that fluoresce green under ultraviolet (UV) illumination. The cultures were also checked for contamination by inoculating a sample of the culture into 9 mL BY media supplemented with 0.1 mL of 0.5 M glucose and incubating at 39° C. for one day. If no turbidity was seen after 1 day, then the culture was considered uncontaminated. Further verification was conducted from time to time by extracting the genomic DNA from the methanogen strain culture and PCR amplifying the 16S rRNA gene, using both the conventional bacterial 16S primers (27f—GAGTTTGATCMTGGCTCAG, 1492r—GGYTACCTTGTTACGACTT) and the archaeal-specific 16S primers (915af—AGGAATTGGCGGGGGAGCAC, 1386r—GCGGTGTGTGCAAGGAGC). The presence of a band with the archaeal primer set and the absence of a band with the bacterial primer set, and the sequencing results of PCR products, were used to validate culture purity.

### 1.1.2 Preparation of Strains to be Tested

Cultures of L. rhamnosus HN001, and control strains (L. plantarum ATCC 8014, L. bulgaricus ATCC 11842) were grown overnight in MRS broth (Sigma-Aldrich) at 39° C. The optical density at 600 nm (OD₆₀₀) was measured for each culture, and a sample of each was serially diluted through MRS medium and the dilutions plated onto MRS agar plates to determine viable counts. For each bacterial culture to be tested, 3 mL of the overnight culture was removed anaerobically from the tube using a 5 mL disposable syringe fitted with a 21G needle, along with 1 mL of CO₂ from the culture headspace. The used needle on the syringe was replaced with a Millex 33 mm filter (0.22 μm; Merck Millipore) and a fresh 21G needle attached. The 1 mL of CO₂ was pushed out through the filter and new needle to flush them with the CO₂ from the headspace and make them anaerobic. The needle was then inserted into a sterile, CO₂-flushed Hungate tube and the culture filtrate was pushed through the filter into the tube. Once prepared, the filtrates in the Hungate tubes were placed into an anaerobic chamber. All of the assay components were assembled in the anaerobic chamber as indicated in Table 1. Multiwell, 96 well plates were then placed into an AnaeroPack 2.5 L Rectangular Jar along with an Anaeropack-Anaero Anaerobic Gas Generator. The lid was sealed, the jar removed from the anaerobic chamber and incubated at 39° C. The plate was observed daily through the transparent jar, and when the methanogen strain control had visible turbidity, the plate was removed from the jar and the optical density (OD₆₀₀) was recorded after 5 seconds shaking in a SpectraMax plate reader. The absorbance readings of the media control wells were subtracted as background, and the % inhibition of methanogen strain growth caused by the filtrate samples, relative to the positive growth control wells, was calculated.

TABLE 1
Plate setup for the Methanogen bioassay.

| | Volumes added (final concentration) in 250 μL per well | | | | | |
|---|---|---|---|---|---|---|
| | | | L. plantarum/ | | | L. rhamnosus |
| | Media | Methanogen | L. bulgaricus | | | HN001 |
| Component | control | alone control | filtrate control | Nisin | | filtrate |
| Media (MRS) | 75 μL (30% | 75 μL (30% | 0 | 65 | μL | 0 |
| | v/v) | v/v) | | | | |
| Nisin (from | 0 | 0 | 0 | 10 μL (12 | | 0 |
| Lactococcus | | | | μM) | | |
| lactis; Sigma | | | | | | |
| Aldrich; | | | | | | |
| 1 mg/ml, 300 μM) | | | | | | |
| Filtered | 0 | 0 | 75 μL (30% | 0 | | 75 μL (30% |
| supernatant | | | v/v) | | | v/v) |
| Phosphate buffer | 5 μL (20 | 5 μL (20 | 5 μL (20 | 5 μL (20 | | 5 μL (20 |
| (0.5M K₂HPO₄, | mM) | mM) | mM) | mM) | | mM) |
| 0.5M KH₂PO₄, | | | | | | |
| pH 7) | | | | | | |
| Methanogen | 0 | 10 μL (4% | 10 μL (4% | 10 μL (4% | | 10 μL (4% |
| inoculum | | v/v) | v/v) | v/v) | | v/v) |
| BY medium | 155 μL | 145 μL | 145 μL | 145 | μL | 145 μL |
| Ethanol | 5 μL (200 | 5 μL (200 | 5 μL (200 | 5 μL (200 | | 5 μL (200 |
| (10M) | mM) | mM) | mM) | mM) | | mM) |
| Vitamin/CoM | 5 μL (2% | 5 μL (2% | 5 μL (2% | 5 μL (2% | | 5 μL (2% |
| solution | v/v) | v/v) | v/v) | v/v) | | v/v) |
| Formate | 5 μL (60 | 5 μL (60 | 5 μL (60 | 5 μL (60 | | 5 μL (60 |
| (3M) | mM) | mM) | mM) | mM) | | mM) |
| TOTAL | 250 μL | 250 μL | 250 μL | 250 | μL | 250 μL |

### 1.2 Results

The L. rhamnosus HN001 culture grew well on MRS broth, attaining an OD₆₀₀ of 4.97 after 16 hours growth. Viable counts from plating of dilutions of the culture onto MRS plates indicated 4.8×10⁹ CFU·mL⁻¹ of culture. These growth parameters were similar to the control strains L. plantarum 8014 and L. bulgaricus 11842, although L. plantarum 8014 had a lower viable count. Filtrates from the test strains were included in the methanogen bioassay, and the plate was incubated at 39° C. for 5 days before being removed from the jar and the OD₆₀₀ of the wells recorded. The readings of the test wells were compared with those of the methanogen strain without any treatment and the % inhibition of growth is shown in Table 2.

TABLE 2
L. rhamnosus HN001 culture filtrate screening
against the indicator methanogen strain.

| | OD₆₀₀ of | | % | | | |
|---|---|---|---|---|---|---|
| | test | | methanogen | Std | % | |
| Treatment | culture | CFU/mL | growth* | Dev | inhibition | P |
| L. plantarum | 4.78 | 2.8E+08 | 91.1 | 3.9 | 8.9 | <0.001 |
| 8014 | | | | | | |
| L. bulgaricus | 4.85 | 2.1E+09 | 106.9 | 11.0 | −6.9 | 0.06 |
| 11842 | | | | | | |
| L. rhamnosus | 4.97 | 4.8E+09 | 77.1 | 14.8 | 22.9 | <0.001 |
| HN001 | | | | | | |
| Nisin (4 μM) | NA | NA | 12.2 | 8.0 | 87.8 | <0.001 |

*% inhibition was calculated from the mean of OD₆₀₀ readings from 16 wells per treatment.

The L. rhamnosus HN001 filtrate significantly reduced the growth of the methanogen strain, on average by nearly 23%. This growth inhibition was higher than seen with either of the control strains, L. plantarum 8014 or L. bulgaricus 11842, which reduced growth by around 9% or had no effect, respectively. The L. rhamnosus HN001 filtrate showed inhibitory activity approximately 25% of the 4 μM nisin control treatment.

### 1.3 Discussion

M. boviskoreani JH1 was used as the indicator strain because Methanobrevibacter spp. make up the majority of methanogens in the rumen across multiple ruminant species. The methanogen inhibitory activity observed in the testing of culture supernatants of L. rhamnosus HN001 was greater than the inhibition seen with the two control strains.

### 1.4 Conclusion

The screening of L. rhamnosus HN001 culture supernatant in a plate-based assay demonstrated an inhibition of the indicator methanogen strain. In relation to control LAB strains, this inhibitory activity was greater than that observed with L. plantarum 8014 or L. bulgaricus 11842 but less than a purified nisin control.

### 2. Example 2—Plate-Based Screening of Lacticaseibacillus rhamnosus HN001 Bacteriocin Extracts and Culture Supernatants Against an Indicator Methanogen Strain

### 2.1 Materials and Methods

### 2.1.1 Methanogen Cultures

Cultures of the indicator methanogen strain were prepared according to item 1.1.1 of Example 1.

### 2.1.2 Cell and Supernatant Samples of L. rhamnosus HN001

Cell and supernatant samples of L. rhamnosus HN001 were derived from a commercial manufacturing run of ˜5,000 L under standard production conditions. Four samples were used, consisting of:
Sample 1. L. rhamnosus HN001 cell sample, unwashed, freeze-dried, low pH extract.
Sample 2. L. rhamnosus HN001 supernatant sample, evaporated @50° C., 35% solids, ˜8× concentrated.
Sample 3. L. rhamnosus HN001 supernatant sample, evaporated @50° C., 45%+solids; ˜11× concentrated.
Sample 4. L. rhamnosus HN001 supernatant sample, freeze-dried.

These samples were kept frozen at −20° C. until needed. Prior to bioassay, the cell sample (Sample 1) was resuspended at 5% (wt/vol) in 0.9% NaCl, pH 6.8, and processed using the bacteriocin extraction method described in 2.1.3 below, resulting in an extract which was stored frozen at −20° C. until use. The evaporated supernatant samples (Samples 2 & 3) and the freeze-dried supernatant sample (Sample 4) were also resuspended at 5% (wt/vol) in 0.9% NaCl, pH 6.8 and filtered through Millex-GP 0.22 μm 25 mm diameter sterile filters (Millipore, Merck, Sigma-Aldrich NZ) into sterile N₂-flushed Hungate tubes using 10 mL syringes. The headspace of the Hungate tubes were further flushed for 30 min with a sterile flow of N₂ to remove traces of O₂ from the samples.

Heat treatments were carried out with the L. rhamnosus HN001 supernatant which generated a further three samples as follows:
Sample 5. L. rhamnosus HN001 supernatant sample, heated at 72° C./15 sec.
Sample 6. L. rhamnosus HN001 supernatant sample, heated at 75° C./4 min.
Sample 7. L. rhamnosus HN001 supernatant sample, heated at 100° C./4 min.

After heat treatment, the samples were filtered through 0.22 μm filters as described above into sterile N₂-flushed Hungate tubes and the headspace flushed with a sterile flow of N₂. The samples were stored at −20° C. until screening in the indicator methanogen bioassay.

### 2.1.3 Bacteriocin Extraction

The resuspended culture was transferred into a 15 mL Falcon tube and the pH adjusted to ˜6.8 with 6M NaOH. The pH-adjusted culture sample was incubated at 70° C. for 45 minutes, then centrifuged for 15 min at 2600×g at 4° C. The supernatant was decanted and the cell pellet was resuspended in 8 mL 0.9% NaCl, pH 2. The pH of the resuspended pellet was checked and if necessary, 1M HCl was used to adjust the final pH to 2. The cells were incubated for 2 hours at 4° C. with slow agitation on a shaking platform. The cells were then centrifuged at 2600×g for 15 min at 4° C., and the supernatant collected into a fresh 15 mL Falcon tube. The pH of the supernatant was adjusted 6.8 with 1M NaOH. The pH-adjusted supernatant was filtered through a Millex-GP 0.22 μm 25 mm diameter sterile filter (Millipore, Merck, Sigma-Aldrich NZ) into a sterile N₂-flushed Hungate tube using a 10 mL syringe, under sterile conditions. The filtered supernatant was frozen at −20° C. until use.

### 2.1.4 Methanogen Bioassay

The bacteriocin extracts contained under anaerobic conditions in Hungate tubes were placed into an anaerobic chamber and allowed to thaw. All of the assay components except the bacteriocin extracts, were added and mixed in a sterile Hungate tube inside the anaerobic chamber in proportion to those indicated in Table 3 and then dispensed into wells of a multiwell 96 well plate containing the bacteriocin extracts. Initial runs of the bioassay used phosphate buffer, however this was found to produce a precipitate, likely due to CaCl₂ in the L. rhamnosus HN001 culture medium reacting with the phosphate buffer to form insoluble calcium phosphate precipitates. As an alternative, the phosphate buffer was replaced with 3-(N-morpholino) propanesulfonic acid (MOPS, 90 mM pH 7.0) in the bioassay.

The plates were then placed into an AnaeroPack 2.5 L Rectangular Jar along with an Anaeropack-Anaero Anaerobic Gas Generator. The lid was sealed, the jar removed from the anaerobic chamber and incubated at 39° C. The plate was observed daily through the transparent jar, and when the methanogen strain control had visible turbidity, the plate was removed from the jar and the optical density of each well was recorded at 600 nm (OD₆₀₀) after 5 seconds shaking in a Spectra Max plate reader. The absorbance readings of the media control wells were subtracted as background, and the % inhibition of growth of the indicator methanogen strain caused by the bacteriocin extract samples, relative to the positive growth control wells (which contained buffer alone), was calculated.

TABLE 3
Microtitre plate setup for the Methanogen bioassay.

| | Volumes added (final concentration) per well | | | | |
|---|---|---|---|---|---|
| | Media | Methanogen | | | L. rhamnosus |
| Component | control | control | Nisin | | HN001 extract |
| Media (MRS) | 75 μL (30% | 75 μL (30% | 65 | μL | 0 |
| | v/v) | v/v) | | | |
| Nisin (1 mg/ | 0 | 0 | 10 μL (12 | | 0 |
| ml, 300 μM) | | | μM) | | |
| Bacteriocin | 0 | 0 | 0 | | 75 μL (30% |
| extract | | | | | v/v) |
| MOPS buffer | 5 μL (90 | 5 μL (90 | 5 μL (90 | | 5 μL (90 |
| (0.1M, pH 7.0) | mM) | mM) | mM) | | mM) |
| Methanogen | 0 | 10 μL (4% | 10 μL (4% | | 10 μL (4% |
| inoculum | | v/v) | v/v) | | v/v) |
| BY medium | 155 μL | 145 μL | 145 | μL | 145 μL |
| Ethanol (10M) | 5 μL (200 | 5 μL (200 | 5 μL (200 | | 5 μL (200 |
| | mM) | mM) | mM) | | mM) |
| Vitamin/CoM | 5 μL (2% | 5 μL (2% | 5 μL (2% | | 5 μL (2% |
| solution | v/v) | v/v) | v/v) | | v/v) |
| Formate (3M) | 5 μL (60 | 5 μL (60 | 5 μL (60 | | 5 μL (60 |
| | mM) | mM) | mM) | | mM) |
| TOTAL | 250 μL | 250 μL | 250 | μL | 250 μL |

### 2.2 Results

The L. rhamnosus HN001 cell bacteriocin extract and the supernatant samples prepared from a commercial manufacturing run were allowed to thaw prior to addition to the MOPS buffer-modified indicator methanogen assays. The L. rhamnosus HN001 sample gave results with large standard deviations in the initial assay and the assay was repeated to get better reproducibility (Table 4).

TABLE 4
L. rhamnosus HN001 samples tested against indicator methanogen strain.

| | Assay 1 | | | | Assay 2 | | | |
|---|---|---|---|---|---|---|---|---|
| L. rhamnosus | Mean % | | | | Mean % | | | |
| HN001 | Growth | Mean % | | | Growth | Mean % | | |
| sample* | JH1 | Inhibition | SD | P | JH1 | Inhibition | SD | P |
| 1. cell sample | 12.9 | 87.1 | 8 | 2.0E−10 | 63.7 | 36.3 | 9 | 0.0014 |
| 2. SN 35% | 67.6 | 32.4 | 5.4 | 1.8E−06 | 53.6 | 46.4 | 17.1 | 0.00066 |
| 3. SN 45% | 65.2 | 34.8 | 6.9 | 8.8E−07 | 53.9 | 46.1 | 11.3 | 0.00025 |
| 4. SN FD | 54 | 46 | 21.8 | 0.0001 | 47.8 | 52.2 | 8.8 | 5.0E−05 |
| 5. SN 72° C. | 53.8 | 46.2 | 21 | 3.2E−05 | 12.5 | 87.5 | 13.6 | 4.2E−07 |
| 6. SN 75° C. | 59.3 | 40.7 | 7.8 | 7.9E−10 | 25.3 | 74.7 | 8 | 1.3E−06 |
| 7. SN 100° C. | 55.3 | 44.7 | 6.4 | 7.0E−11 | 2.3 | 97.7 | 7.5 | 1.3E−07 |
| Nisin | 5.2 | 97.5 | 1.6 | 4.2E−11 | 18.2 | 81.8 | 10 | 1.3E−06 |

*Bacteriocin extractions from the cell sample (1) is expressed as % growth of the indicator methanogen strain with 0.9% NaCl; Supernatant samples (2-7) are expressed as % growth of the indicator methanogen strain in MRS + NH₄ lactate (MRS medium with 4% lactic acid neutralized with ammonium hydroxide) used as a control to mimic the culture supernatant composition from the commercial manufacturing run.

### 2.3 Conclusion

Both the L. rhamnosus HN001 cell bacteriocin extract and the supernatant samples showed strong inhibition of growth of the indicator methanogen strain, as did the nisin control. Moreover, the inhibitory activity of the supernatant samples was retained following freeze-drying, as well as heating of the supernatant. Based on these results the applicant considers that the HN001 inhibitory activity withstands common conditions associated with processing, such as spray drying.

### 3. Example 3—Rumen In Vitro Model for Testing the Effects of Lacticaseibacillus rhamnosus HN001 on a Simulated Rumen Microbial Community

### 3.1 Materials and Methods

### 3.1.1 Preparation of L. rhamnosus HN001 Cultures and Supernatants for Testing

L. rhamnosus HN001 was inoculated into 7 Hungate tubes containing 5 mL of anaerobic MRS medium (Sigma-Aldrich) and were incubated at 39° C. for 16 hours (until the cultures reached stationary phase). All the cultures were pooled into a 250 mL CO₂-flushed serum bottle. An aliquot (1 mL) of the combined cultures was added to 9 mL of sterile MRS medium to measure its OD₆₀₀. Further aliquots (0.5 mL) of the culture mix were inoculated in triplicate into 4.5 mL of sterile anaerobic buffer and serially 10-fold diluted under CO₂ and plated onto MRS plates to determine the number of colony forming units (CFU·mL⁻¹) of original culture. Half of the remaining culture was used for one set of rumen in vitro fermentations (L. rhamnosus HN001 culture) and the other half was filtered (Millipore 0.22 μm pore size) and the filtrate was placed into a new sterile anaerobic serum bottle (L. rhamnosus HN001 supernatant treatment, SN). Anaerobic phosphate buffer (0.46 M K₂HPO₄; 0.54 M KH₂PO₄, pH 7) was used as the no treatment control (Buffer).

### 3.1.2 Rumen Fluid Preparation and In Vitro Fermentation Set Up

For inoculation of the rumen in vitro fermentation vessels, fresh rumen contents were collected from 6 rumen-fistulated Friesian cows. After squeezing through 1 layer of cheesecloth, the resulting rumen fluids from two animals were combined (approx. 150 mL rumen fluid) giving 3 biological replicates. Aliquots (12.5 mL) of the mixed rumen fluid were added to 0.5 mg dried grass and 36.5 mL of anaerobic phosphate buffer in a 250 mL serum bottle. The treatments (1 mL) of either Buffer, L. rhamnosus HN001 or SN were added before closing the serum bottles with butyl rubber stoppers, giving a final fermentation volume of 50 mL containing 25% rumen fluid (v/v). The experimental layout of the treatment and control bottles is shown in Table 5.

TABLE 5
Number of bottles inoculated with L. rhamnosus HN001 culture or
supernatant (SN) or control solution (Buffer) for each replicate,
used for volatile fatty acid (VFA)/DNA sampling and analysis.

| | Bottles | | |
|---|---|---|---|
| | VFA and DNA sampling | | |
| Treatment | Rep 1 | Rep 2 | Rep 3 |
| L. rhamnosus HN001 | 2 | 2 | 2 |
| SA | 2 | 2 | 2 |
| Buffer | 2 | 2 | 2 |
| TOTAL | 6 | 6 | 6 |

### 3.1.3 VFA Sample Collections and Analyses

Samples were collected for VFA analysis from bottles at 0, 2, 6, 12, 24, 48 hours. At each time point, 3 mL aliquots were collected, and their pH measured. The samples were divided into a 0.9 mL sub-sample for DNA analysis, and 1.8 mL for VFA and non-VFA analyses (including lactic acid). VFA samples were centrifuged at 21,000×g for 10 min at 4° C. and 0.9 mL of supernatant was removed and added to 0.1 mL of internal standard (20 mM 2-ethylbutyrate in 20% phosphoric acid), mixed and frozen at −20° C. until analysis. After thawing and re-centrifugation at 21,000×g for 10 min at 4° C., 0.9 mL was collected for derivatization for non-VFA analysis, while the remainder of the sample was analysed directly via GC.

### 3.1.4 DNA Sample Collection and Analyses

DNA samples were collected at the same intervals as the VFA samples and were immediately frozen at −20° C. until DNA extraction. DNA was extracted using a bead-beating/phenol chloroform method (Rius et al., 2012) and used in PCR reactions to generate 16S ribosomal RNA gene amplicons with barcoded sequencing primers specific for bacteria, archaea and protozoa (Kittelmann and Janssen, 2011). Amplicons were purified, normalised, pooled and sequenced via an Illumina MiSeq sequencer. Sequencing results were quality controlled and filtered and the filtered sequences were analysed via QIIME using the Silva database with rumen-specific 16S rRNA gene sequences. Operational taxonomic units (OTUs) were picked at 99% similarity and tabulated.

### 3.2 Results

### 3.2.1 Rumen In Vitro pH

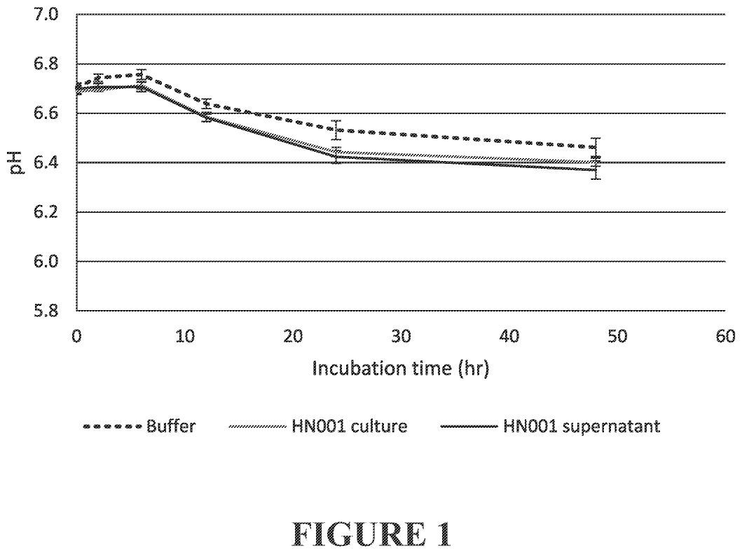

A decrease in pH value was observed during the course of the in vitro incubation, consistent with normal fermentation and the accumulation of short chain fatty acids. The addition of L. rhamnosus HN001 or SN both induced decreased pH compared to the buffer control (p<0.01). This difference appeared 2 hours after the beginning of incubation and persisted until the end of the assay (FIG. 1). The overall decrease in pH from 0 to 48 hours represents around 0.3 pH units with a maximum difference between treatments and control of 0.1 observed after 24 hours of incubation.

### 3.2.2 Impact of L. rhamnosus HN001 on Rumen In Vitro VFA and Lactic Acid Production

The total amount of VFA produced in the rumen in vitro fermentations increased during the incubation, indicating the fermentation of substrates via the activities of the ruminal microbes in the inoculum (Table 6). Between 6 hours and 24 hours, higher amounts of VFAs were measured in bottles inoculated with L. rhamnosus HN001 or SN, compared to the bottles receiving Buffer only.

TABLE 6
Total VFA production during rumen in vitro fermentations
inoculated with L. rhamnosus HN001, SN, or Buffer.
Total VFA [mM]

| Incubation time | | L. rhamnosus | |
|---|---|---|---|
| (hours) | Buffer | HN001 | SN |
| 0 | 17 ± 1 | 18 ± 1 | 18 ± 1 |
| 2 | 25 ± 1 | 27 ± 2 | 26 ± 2 |
| 6 | 33 ± 1 | 40 ± 1*** | 40 ± 1*** |
| 12 | 51 ± 1 | 57 ± 1*** | 58 ± 2*** |
| 24 | 71 ± 2 | 77 ± 1*** | 77 ± 1*** |
| 48 | 84 ± 2 | 87 ± 3 | 88 ± 5 |

T-test L. rhamnosus HN001/SN vs Buffer
***p < 0.001.

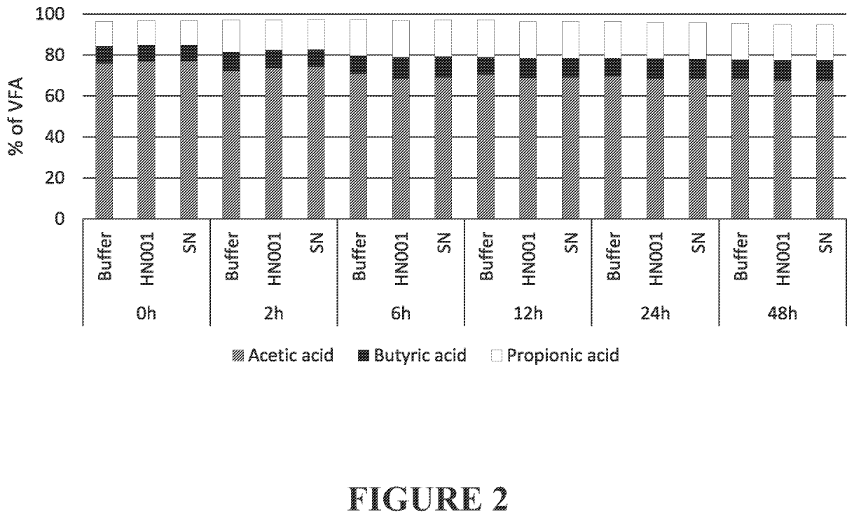

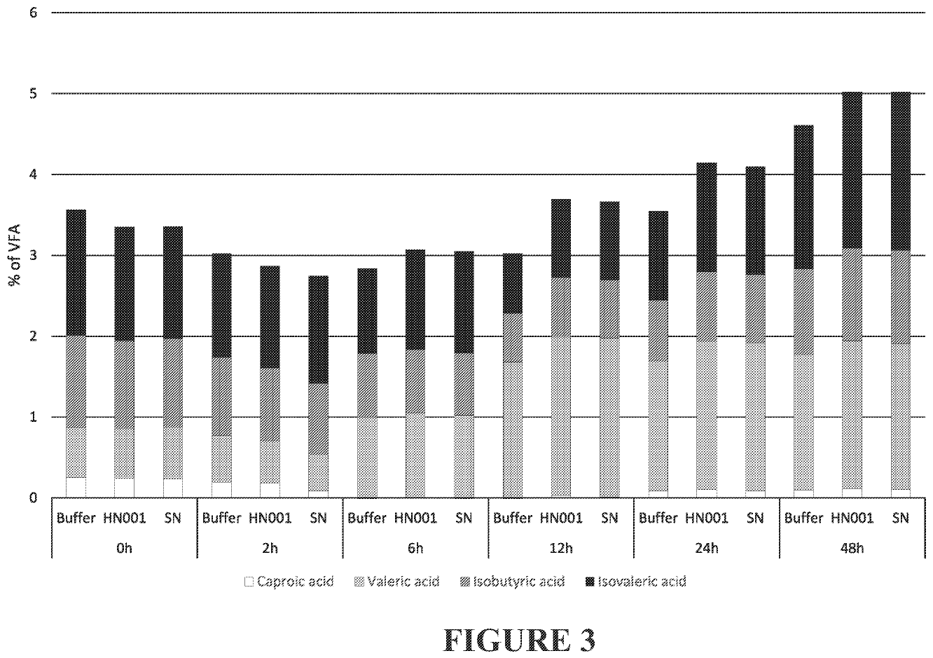

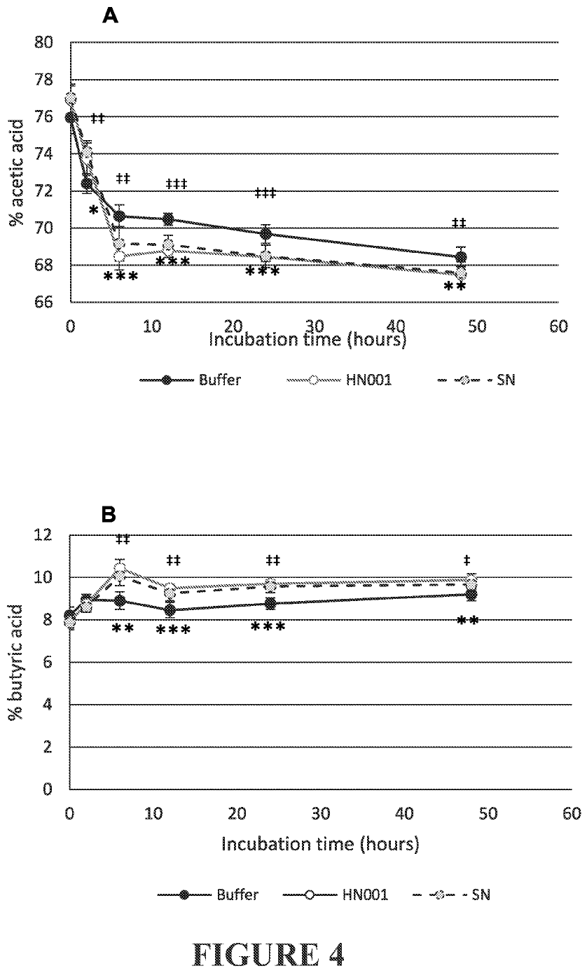

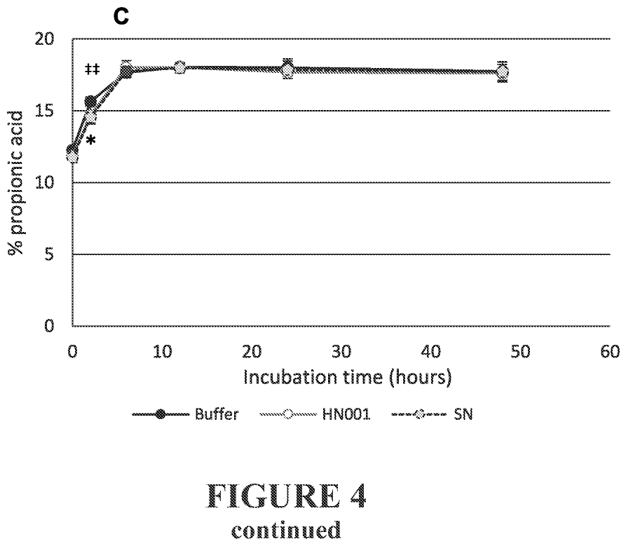

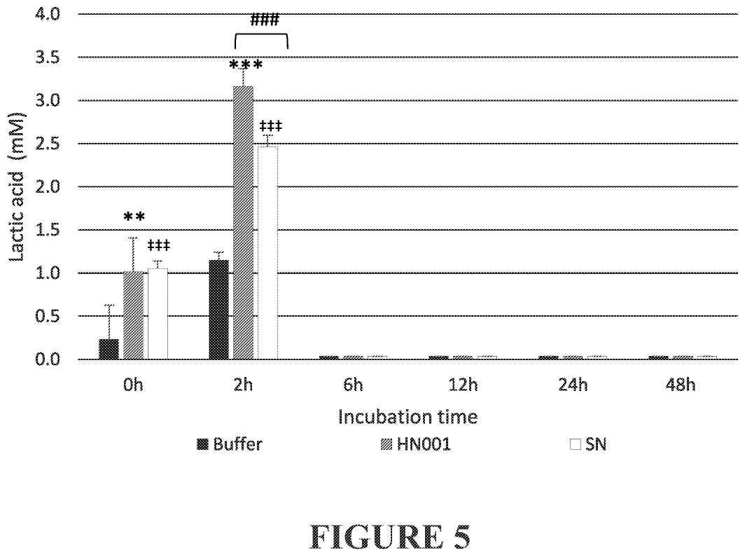

Acetic acid was the main VFA produced for all of the fermentations, followed by propionic acid and butyric acid (FIG. 2). The proportion of minor VFAs is shown in FIG. 3. During incubation, all of the in vitro fermentations showed a decrease in acetic acid proportion and an increase in butyric acid proportions between 0 and 6 hours. After 6 hours, the proportion of VFAs remained stable until the end of fermentations. The proportion of propionic acid showed a slight increase at 6 hours and remained stable until 48 hours (FIG. 4). Addition of L. rhamnosus HN001 or SN induced a slight increase in acetic acid at 2 hours, then decreased acetic acid production from 4 hours until 48 hours compared to the Buffer control (FIG. 4). Concomitantly, the proportion of butyric acid increased in the fermentations receiving the L. rhamnosus HN001 or SN from 6 hours until the end of the experiment. Compared to buffer, propionic acid levels were decreased slightly (but significantly) for the L. rhamnosus HN001 culture and supernatant treatments only at 2 hours of fermentation (p=0.012 Buffer vs L. rhamnosus HN001; p=0.0009 Buffer vs SN).

The acetic/propionic acid (A:P) ratio increased at 2 hours of incubation for L. rhamnosus HN001 and SN bottles compared to Buffer control (Table 7). At 6 hours, the A:P ratio was decreased for the HN001 group, but no differences were observed between SN and Buffer control.

TABLE 7
Acetic acid/propionic acid ratio in supernatants from rumen in
vitro bottles inoculated with L. rhamnosus HN001, SN, or Buffer.
Acetic/propionic acid ratio

| Incubation time | | L. rhamnosus | |
|---|---|---|---|
| (hours) | Buffer | HN001 | SN |
| 0 | 6.2 ± 0.3 | 6.5 ± 0.3 | 6.6 ± 0.3 |
| 2 | 4.6 ± 0.1 | 5.0 ± 0.3* | 5.1 ± 0.2^{‡‡‡} |
| 6 | 4.0 ± 0.1 | 3.8 ± 0.1* | 3.9 ± 0.2 |
| 12 | 3.9 ± 0.1 | 3.8 ± 0.1 | 3.8 ± 0.1 |
| 24 | 3.9 ± 0.2 | 3.9 ± 0.1 | 3.8 ± 0.2 |
| 48 | 3.9 ± 0.2 | 3.8 ± 0.1 | 3.8 ± 0.2 |

T-test L. rhamnosus HN001 vs Buffer
*p < 0.05;
SN vs Buffer;
^{‡‡‡}p < 0.001.

Lactic acid is a major fermentation product from lactic acid bacteria (LAB) and can be produced in the rumen. In the rumen in vitro fermentations, lactic acid was seen at ˜1 mM at 0 hours in the bottles receiving L. rhamnosus HN001 or SN due to the presence of lactic acid (˜46.5 mM) produced in the L. rhamnosus culture used as the source of the inoculum and supernatant. However, the lactic acid concentration in the L. rhamnosus HN001 or SN bottles increased to ˜2.5-3 mM after 2 hours of fermentation, suggesting that lactic acid was produced during these fermentations. Lactic acid rapidly disappeared from 6 hours onwards, suggesting that it was subsequently metabolised by rumen microbes.

### 3.2.3 Rumen Microbial Community Composition

### 3.2.3.1. Bacteria

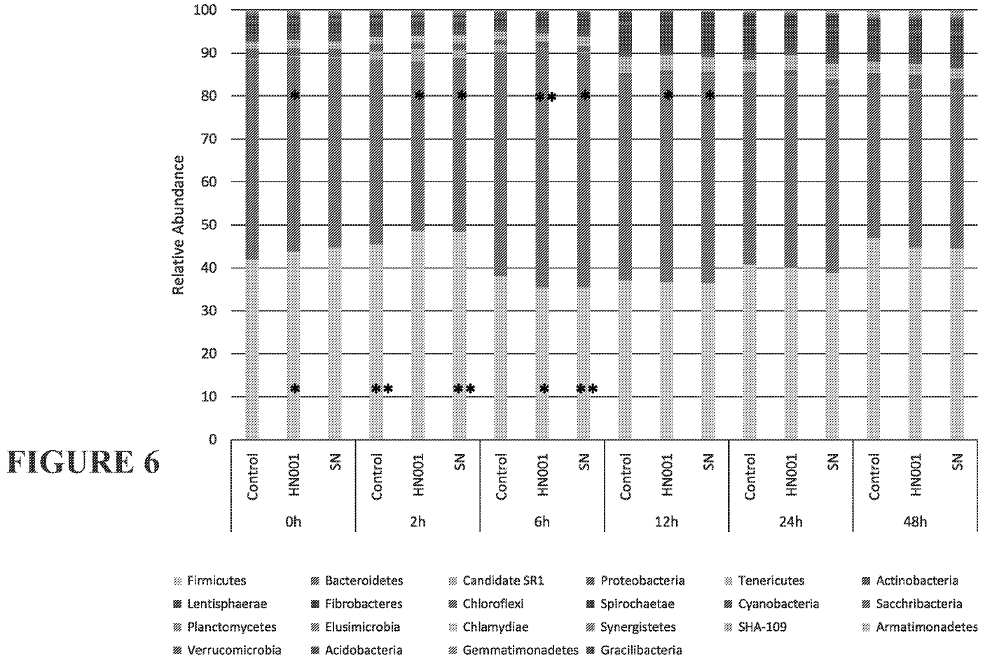

At the phylum level, Firmicutes and Bacteroidetes were the major phyla identified in the rumen in vitro samples, regardless of the treatment (FIG. 6). The balance between these two phyla shifted during the fermentation runs; Firmicutes were dominant at 6 hours (p<0.01) and 48 hours, while Bacteroidetes became dominant at 12 hours. The L. rhamnosus HN001 and SN had the same impact on Firmicutes and Bacteroidetes proportions, leading to an increased proportion of Firmicutes (p<0.01) and a decrease in Bacteroidetes (p<0.05) compared to the Buffer control group after 2 hours of incubation. At 6 hours, this balance was inversed; Firmicutes become less abundant (L. rhamnosus HN001, p<0.05; SN, p<0.01) while Bacteroidetes increased (L. rhamnosus HN001, p<0.01; SN, p<0.05) in L. rhamnosus HN001 and SN groups compared to the Buffer control. The addition of L. rhamnosus HN001 or SN also influenced other phyla such as Candidate SR1 (decrease at 6 hours (p<0.01), increase at 24 hours (p<0.05)), while Proteobacteria, Tenericutes, and Verrucomicrobia all increased at 24 hours (p<0.05).

Genus-level analyses demonstrated that Prevotella, Ruminococcus, and genera belonging to the Christensenellaceae and Rikenellaceae families and the order Bacteroidales were predominant in the rumen in vitro samples. Shannon Diversity analysis demonstrated an impact of both L. rhamnosus HN001 and SN on the rumen bacterial community (Table 8). As described in FIG. 6, this enrichment in diversity occurred at 2 hours and 24 hours for L. rhamnosus HN001 and SN groups compared to the Buffer control.

TABLE 8
Shannon Diversity analysis of bacterial diversity at the genus level.
Shannon Diversity Index

| Incubation time | | L. rhamnosus | |
|---|---|---|---|
| (hours) | Buffer | HN001 | SN |
| 0 | 3.61 | 3.62 | 3.57 |
| 2 | 3.60 | 3.63* | 3.64* |
| 6 | 3.33 | 3.29 | 3.28 |
| 12 | 3.37 | 3.39 | 3.31 |
| 24 | 3.47 | 3.60* | 3.58* |
| 48 | 3.76 | 3.94 | 3.94 |

T-test vs Buffer
*p < 0.05

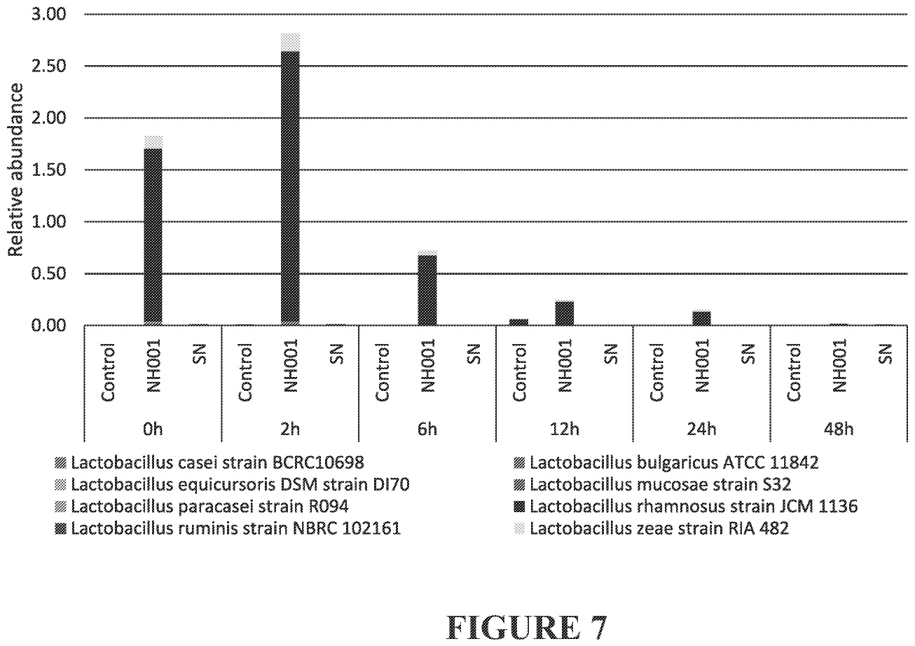

The viable counts of the L. rhamnosus HN001 inoculum added to the rumen in vitro fermentations was 10¹⁰ CFU·mL⁻¹. As anticipated, bacteria associated with the genus Lactobacillus were significantly increased in the L. rhamnosus HN001 group compared to both the SN and Buffer control groups, except at 12 hours. Further identification of Lactobacillus species based on 16S rRNA gene sequence confirmed that the increase in total lactobacilli for the L. rhamnosus HN001 treatment was due mainly to L. rhamnosus (FIG. 7). The proportion of L. rhamnosus in the HN001 group was significantly higher compared to Buffer and the SN group throughout the experiment. Lactobacillus zeae strain RIA 482 was also significantly increased in the L. rhamnosus HN001 group compared to Buffer and SN groups (FIG. 7).

### 3.2.3.2. Archaea

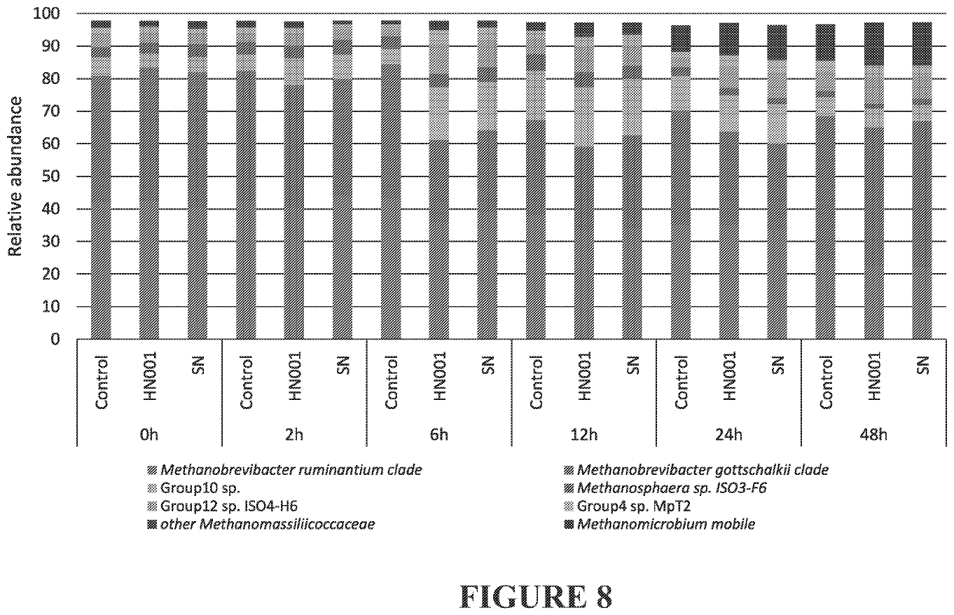

Analysis of Archaea diversity demonstrated important changes in composition of this kingdom at 6 hours and 24 hours (FIG. 8). Mbb. ruminantium and Mbb. gottschalkii were the dominant Archaea detected in the rumen in vitro samples for the duration of the fermentations (FIG. 8). The Shannon Diversity Index was higher for both groups inoculated with L. rhamnosus HN001 culture or supernatant compared to Buffer after 6 hours of incubation (Table 9). For L. rhamnosus HN001 and SN groups, Mbb. boviskoreani, Mbb. gottschalkii, Mbb. ruminantium and Mbb. smithii were significantly lower from 6 to 48 hours compared to buffer (Tables 10-13). After 6 and 12 hours of incubation, Mbb. boviskoreani was significantly lower for the L. rhamnosus HN001 (p<0.05) and SN (p<0.01) groups.

TABLE 9
Shannon Diversity analysis of Archaea at clade level.
Shannon Diversity Index

| Incubation time | | L. rhamnosus | |
|---|---|---|---|
| (hours) | Buffer | HN001 | SN |
| 0 | 1.42 | 1.37 | 1.37 |
| 2 | 1.38 | 1.47 | 1.40 |
| 6 | 1.32 | 1.75*** | 1.68*** |
| 12 | 1.63 | 1.78 | 1.73 |
| 24 | 1.62 | 1.73 | 1.81* |
| 48 | 2.37 | 2.46 | 2.47 |

T-test HN001/SN vs Buffer
*p < 0.05
***p < 0.001

TABLE 10
Relative abundance of Mbb. boviskoreani
in rumen in vitro fermentations.

| Incubation time | | L. rhamnosus | |
|---|---|---|---|
| (hours) | Buffer | HN001 | SN |
| 0 | 0.10 | 0.16 | 0.12 |
| 2 | 0.16 | 0.19 | 0.11 |
| 6 | 0.13 | 0.07* | 0.06** |
| 12 | 0.09 | 0.04* | 0.03** |
| 24 | 0.07 | 0.05 | 0.05 |
| 48 | 0.10 | 0.08 | 0.07 |

T-test HN001/SN vs Buffer
*p < 0.05,
**p < 0.01,
***p < 0.001

TABLE 11
Relative abundance of Mbb. gottschalkii
in rumen in vitro fermentations.

| Incubation time | | L. rhamnosus | |
|---|---|---|---|
| (hours) | Buffer | HN001 | SN |
| 0 | 38.80 | 40.61 | 41.47 |
| 2 | 40.02 | 37.62 | 35.60 |
| 6 | 41.00 | 25.44*** | 25.07*** |
| 12 | 29.44 | 25.49 | 28.58 |
| 24 | 34.95 | 28.73** | 26.84*** |
| 48 | 44.19 | 42.65 | 45.00 |

T-test HN001/SN vs Buffer
*p < 0.05,
**p < 0.01,
***p < 0.001

TABLE 12
Relative abundance of Mbb. ruminantium
in rumen in vitro fermentations.

| Incubation time | | L. rhamnosus | |
|---|---|---|---|
| (hours) | Buffer | HN001 | SN |
| 0 | 42.16 | 42.60 | 40.57 |
| 2 | 42.32 | 40.42 | 44.30 |
| 6 | 43.41 | 35.88** | 39.18 |
| 12 | 37.83 | 33.66 | 34.08 |
| 24 | 35.22 | 35.19 | 33.39 |
| 48 | 24.27 | 22.53 | 21.89 |

T-test HN001/SN vs Buffer
*p < 0.05,
**p < 0.01,
***p < 0.001

TABLE 13
Relative abundance of Mbb. smithii
in rumen in vitro fermentations.

| Incubation time | | L. rhamnosus | |
|---|---|---|---|
| (hours) | Buffer | HN001 | SN |
| 0 | 0.44 | 0.44 | 0.28 |
| 2 | 0.41 | 0.44 | 0.44 |
| 6 | 0.39 | 0.22** | 0.28* |
| 12 | 0.32 | 0.23 | 0.32 |
| 24 | 0.38 | 0.32 | 0.29 |
| 48 | 0.35 | 0.30* | 0.38 |

T-test HN001/SN vs Buffer
*p < 0.05,
**p < 0.01,
***p < 0.001

### 3.2.3.3. Protozoa

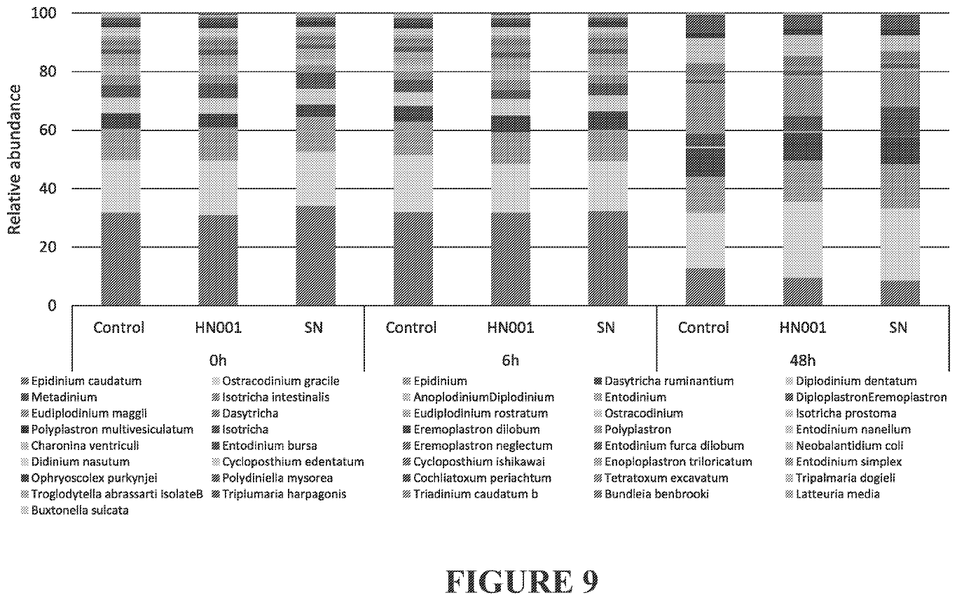

The main protozoa identified in samples were Epidinium and Ostracodinium. Protozoal community analysis revealed changes in diversity after 6 and 48 hours of incubation (FIG. 9). Shannon Diversity Index analysis demonstrated a single significant difference between the buffer control and the L. rhamnosus HN001 culture group at 6 hours (Table 14), with an increase of diversity in the sample inoculated with L. rhamnosus HN001. Protozoal species Polyplastron multivesiculatum and Eremoplastron dilobum were significantly lower (p<0.05) at 6 hours with L. rhamnosus HN001 vs buffer. Protozoa appear to be less affected by the inoculation of L. rhamnosus HN001 or SN compared to Bacteria or Archaea.

TABLE 14
Shannon Diversity analysis of protozoa.
Shannon Diversity Index

| Incubation time | | L. rhamnosus | |
|---|---|---|---|
| (hours) | Buffer | HN001 | SN |
| 0 | 2.32 | 2.32 | 2.19 |
| 2 | 2.37 | 2.34 | 2.30 |
| 6 | 2.27 | 2.34* | 2.30 |
| 12 | 2.25 | 2.30 | 2.28 |
| 24 | 2.21 | 2.19 | 2.18 |
| 48 | 2.29 | 2.22 | 2.17 |

T-test vs Buffer
*p < 0.05.

### 3.3 Discussion

Lactic acid bacteria (LAB) are natural inhabitants of the mammalian intestinal tract and are well known for their use in dairy and meat products (Kroeckel, 2006; Ljungh and Wadström, 2006; Zafiriadis, 2015). LAB may influence the rumen ecosystem via several mechanisms; LAB produce organic acids, hydrogen peroxides, non-ribosomal synthesised peptides and bacteriocins (Cotter et al., 2013; Mangoni and Shai, 2011) which are all capable of changing microbial communities. LAB inoculated into silage or directly added to the feed can produce these specific antimicrobial compounds active against other microorganisms.

The use of bacteriocins in livestock feed has also been reported to enhance the growth potential of the host (Yang et al., 2014). Bacteriocin addition to silage is thought to enable the cellulolytic bacteria in the rumen to become more dominant (Capper et al., 2009). Bacteriocins have a number of advantages over other antimicrobial agents, which include target specificity (Lohans and Vederas, 2012), amenability to genetic manipulation (Perez et al., 2014) and a long history of safe use in food for human consumption (Kalmokoff et al., 1996). Callaway et al. (1997) discovered that nisin (>1 μM) added to alfalfa hay reduced the ratio of acetate to propionate production, which was consistent with a decrease in methane generation. An example of a rumen sourced bacteriocin is bovicin HC5, which is produced by Streptococcus bovis HC5, isolated from the bovine rumen. This bacteriocin was found to reduce methane production by 50% in a mixed rumen in vitro assay and ruminal methanogens did not become resistant to the bacteriocin after four transfers in its presence (Lee et al., 2002). Renuka et al. (2013) evaluated the effects of pediocin on in vitro methane production and dry matter digestibility and reported that pediocins P1 and P2 resulted in low levels of methane production (4.81% and 5.08%, respectively) with significant differences reported between active bacteriocin and control groups.

The rumen in vitro assay was conducted to look at the impact of L. rhamnosus HN001 and SN on microbial activity under simulated ruminal fermentation conditions. This assay demonstrated that the addition of L. rhamnosus HN001 or SN to the simulated ruminal ecosystem induced notable changes in VFAs and lactic acid concentrations after 6 hours of incubation. Total VFAs increased, as did butyric acid, while acetic acid decreased. The analysis of the microbial community in the rumen in vitro assays clearly demonstrated changes to both bacterial and archaeal populations in response to the L. rhamnosus HN001 and SN treatments, which are more likely to account for the observed shifts in metabolism.

Predictably, Lacticaseibacillus spp., specifically L. rhamnosus, were enriched in the L. rhamnosus HN001 0 hour sample, which corresponds to the inoculum proportion added to rumen in vitro (10¹⁰ CFU·mL⁻¹). This enrichment was also increased at 2 hours, indicating that L. rhamnosus HN001 grew under rumen-like conditions after inoculation. Furthermore, the lactic acid concentration increased from ˜1 mM at 0 hours (due to carry over of lactic acid from the inoculum) up to ˜3 mM at 2 hours indicating that L. rhamnosus HN001, or other ruminal LABs (e.g. Streptococcus and Lachnospiraceae), were metabolically active, producing additional lactic acid in the rumen fermentations. Despite the strong competition for soluble sugars in the ruminal environment, the L. rhamnosus HN001 strain was able to survive, and remained detectable until 24 hr (0.23% of total bacteria) and was the dominant Lacticaseibacillus strain in the rumen in vitro. This ability to persist and to be active in the simulated ruminal environment demonstrates that L. rhamnosus HN001 can establish a viable population in the rumen, at least in the short term.

The main changes observed within the archaeal communities were the large decreases in the hydrogenotrophic methanogens Mbb. boviskoreani, Mbb. gottschalkii, Mbb. ruminantium and Mbb. smithii from 6 to 48 hours. Because Mbb. gottschalkii and Mbb. ruminantium together make up ˜75% of the archaeal community in the rumen (Doyle et al., 2019), the large decreases observed in these clades is particularly notable.

It should also be noted that the rumen in vitro assays are a closed system and may become nutrient-limited over time. Therefore, the 0-12 hour timepoints may more accurately reflect the situation in vivo, as animals will typically ingest more food and liquid over a 24-hour period.

### 3.4 Conclusion

The rumen in vitro assay of the L. rhamnosus HN001 and SN demonstrated impacts on fermentation end products and on the bacterial and archaeal communities. Overall, the results show that L. rhamnosus HN001 has a specific inhibitory effect on rumen methanogens, and resulted in significant changes to the rumen microbiome, mediated by a compound, or compounds (such as a bacteriocin) which is produced by L. rhamnosus HN001 and secreted into the culture supernatant. Significant increases in volatile fatty acids and total VFA in response to both HN001 and SN were identified. This suggests a shift in hydrogen metabolism away from methanogenesis to short chain/volatile fatty acid (VFA) production and/or disruption of cross-feeding of intermediates between members of the microbiome due to changes in the rumen microbiome. hydrogen metabolism from methanogenesis to short chain/volatile fatty acid (VFA) production. The significant increases in total VFA and butyric acid suggests that animal feed efficiency is also likely to be improved.

### 4. Example 4—On-Farm Cell Culture

### 4.1 Materials and Methods

A mixture of cultures of L. rhamnosus HN001™ and Lactobacillus lactis subsp. cremoris 2566 were added to thermalised milk with and without yeast extract (YE) and incubated using a water bath held at 25° C. or 30° C. for 12 hours. Viable cell counts were measured.

### 4.2 Results

L. rhamnosus HN001™ was able to grow well in a thermalised milk medium at either 25° C. or 30° C., achieving viable cell counts in excess of 5×10⁸ cells/g in combined culture with L. lactis subsp. cremoris 2566 (Table 15). The addition of yeast extract (YE) slightly increased the viable cell count of L. rhamnosus HN001™.

TABLE 15
Viable cell counts.

Viable cell count (cells/g)

| | L. rhamnosus | L. lactis subsp. |
|---|---|---|
| Treatment | HN001 | cremoris 2566 |
| 30° C. + YE | 1.95 × 10⁸ | 9.50 × 10⁸ |
| 30° C. | 1.70 × 10⁸ | 1.11 × 10⁹ |
| 25° C. + YE | 5.10 × 10⁸ | 1.35 × 10⁹ |
| 25° C. | 9.50 × 10⁷ | 9.10 × 10⁸ |

### 4.3 Conclusion

This Example shows that L. rhamnosus HN001™ can be cultured to high cell density using a thermalised milk medium, suitable for on-farm applications.

Preferred embodiments of the invention have been described by way of example only and modifications may be made thereto without departing from the scope of the invention.

### REFERENCES

Aguilar-Uscanga, B. R., Solís-Pacheco, J. R., Plascencia, L., Aguilar-Uscanga, M. G., Garcia, H. S., Lacroix, M., 2020. EFFECT OF CULTURE MEDIUM ON BACTERIOCIN PRODUCTION BY LACTOBACILLUS RHAMNOSUS HN001 AND LACTOBACILLUS REUTERI ATCC 53608. J. Microbiol. Biotechnol. Food Sci. 2020/21, 2462-2468.

Callaway, T. R., Carneiro De Melo, A. M. S., Russell, J. B., 1997. The Effect of Nisin and Monensin on Ruminal Fermentations In Vitro. Curr. Microbiol. 35, 90-96. https://doi.org/10.1007/s002849900218

Capper, J. L., Cady, R. A., Bauman, D. E., 2009. The environmental impact of dairy production: 1944 compared with 2007. J. Anim. Sci. 87, 2160-2167. https://doi.org/10.2527/jas.2009-1781

Cotter, P. D., Ross, R. P., Hill, C., 2013. Bacteriocins—a viable alternative to antibiotics? Nat. Rev. Microbiol. 11, 95-105. https://doi.org/10.1038/nrmicro2937

Doyle, N., Mbandlwa, P., Kelly, W. J., Attwood, G., Li, Y., Ross, R. P., Stanton, C., & Leahy, S. (2019). Use of Lactic Acid Bacteria to Reduce Methane Production in Ruminants, a Critical Review. Frontiers in microbiology, 10, 2207. https://doi.org/10.3389/fmicb.2019.02207

Dimitrijević, R., Stojanović, M., Zivković, I., Petersen, A., Jankov, R. M., Dimitrijević, L., Gavrović-Jankulović, 2009. The identification of a low molecular mass bacteriocin, rhamnosin A, produced by Lactobacillus rhamnosus strain 68. J. Appl. Microbiol. 107, 2108-2115. https://doi.org/10.1111/j.1365-2672.2009.04539.x

IPCC, 2014. Mitigation of Climate Change. Contribution of Working Group III to the Fifth Assessment Report of the Intergovernmental Panel on Climate Change. Cambridge University Press, Cambridge.

Jackson, R. B., Saunois, M., Bousquet, P., Canadell, J. G., Poulter, B., Stavert, A. R., Bergamaschi, P., Niwa, Y., Segers, A., Tsuruta, A., 2020. Increasing anthropogenic methane emissions arise equally from agricultural and fossil fuel sources. Environ. Res. Lett. 15, 071002. https://doi.org/10.1088/1748-9326/ab9ed2

Janssen, P. H., Schuhmann, A., Mörschel, E., Rainey, F. A., 1997. Novel anaerobic ultramicrobacteria belonging to the Verrucomicrobiales lineage of bacterial descent isolated by dilution culture from anoxic rice paddy soil. Appl. Environ. Microbiol. 63, 1382-1388. https://doi.org/10.1128/AEM.63.4.1382-1388.1997

Joblin, K. N., 2005. Methanogenic archaea, in: Makkar, H. P. S., McSweeney, C. S. (Eds.), Methods in Gut Microbial Ecology for Ruminants. Springer Netherlands, Dordrecht, pp. 47-53. https://doi.org/10.1007/1-4020-3791-0_4

Kalmokoff, M. L., Bartlett, F., Teather, R. M., 1996. Are ruminal bacteria armed with bacteriocins? J. Dairy Sci. 79, 2297-2306. https://doi.org/10.3168/jds.S0022-0302(96)76608-0

Kamke, J., Kittelmann, S., Soni, P., Li, Y., Tavendale, M., Ganesh, S., Janssen, P. H., Shi, W., Froula, J., Rubin, E. M., Attwood, G. T., 2016. Rumen metagenome and metatranscriptome analyses of low methane yield sheep reveals a Sharpea-enriched microbiome characterised by lactic acid formation and utilisation. Microbiome 4, 56. https://doi.org/10.1186/s40168-016-0201-2

Kittelmann, S., Janssen, P. H., 2011. Characterization of rumen ciliate community composition in domestic sheep, deer, and cattle, feeding on varying diets, by means of PCR-DGGE and clone libraries. FEMS Microbiol. Ecol. 75, 468-481. https://doi.org/10.1111/j.1574-6941.2010.01022.x

Kroeckel, L., 2006. Use of probiotic bacteria in meat products. Fleischwirtschaft 86, 109-113.

Lee, S. S., Hsu, J.-T., Mantovani, H. C., Russell, J. B., 2002. The effect of bovicin HC5, a bacteriocin from Streptococcus bovis HC5, on ruminal methane production in vitro. FEMS Microbiol. Lett. 217, 51-55. https://doi.org/10.1111/j.1574-6968.2002.tb11455.x

Ljungh, A., Wadström, T., 2006. Lactic acid bacteria as probiotics. Curr. Issues Intest. Microbial. 7, 73-89.

Lohans, C. T., Vederas, J. C., 2012. Development of Class IIa Bacteriocins as Therapeutic Agents. Int. J. Microbiol. 2012, 386410. https://doi.org/10.1155/2012/386410

Mangoni, M. L., Shai, Y., 2011. Short native antimicrobial peptides and engineered ultrashort lipopeptides: similarities and differences in cell specificities and modes of action. Cell. Mol. Life Sci. CMLS 68, 2267-2280. https://doi.org/10.1007/s00018-011-0718-2

Perez, R. H., Zendo, T., Sonomoto, K., 2014. Novel bacteriocins from lactic acid bacteria (LAB): various structures and applications. Microb. Cell Factories 13 Suppl 1, S3. https://doi.org/10.1186/1475-2859-13-S1-S3

Renuka, Puniya, M., Sharma, A., Malik, R. K., Upadhyay, R. C., Puniya, A. K., 2013. Influence of pediocin and enterocinon in-vitro methane, gas production and digestibility. Int.J.Curr.Microbiol.App.Sci. 2, 132-142.

Rius, A. G., Kittelmann, S., Macdonald, K. A., Waghorn, G. C., Janssen, Sikkema, E., 2012. Nitrogen metabolism and rumen microbial enumeration in lactating cows with divergent residual feed intake fed high-digestibility pasture. J. Dairy Sci. 95, 5024-5034. https://doi.org/10.3168/jds.2012-5392

Tyrrell, H. F., Reid, J. T., 1965. Prediction of the energy value of cow's milk. J. Dairy Sci. 48, 1215-1223. https://doi.org/10.3168/jds.S0022-0302(65)88430-2

Yang, S.-C., Lin, C.-H., Sung, C. T., Fang, J.-Y., 2014. Antibacterial activities of bacteriocins: application in foods and pharmaceuticals. Front. Microbiol. 5. https://doi.org/10.3389/fmicb.2014.00241

Zafiriadis, T., 2015. The Use Of Lactic Acid Microbes In Dairy Industry And Their Role As Probiotics—A Review. https://doi.org/10.13140/RG.2.1.2398.9600

### INDUSTRIAL APPLICABILITY

This invention relates to the use of probiotic bacteria, particularly Lacticaseibacillus rhamnosus strain HN001 or derivatives thereof, and in particular the use to inhibit the growth of methane-producing bacteria in the forestomach of ruminant animals and/or to reduce the ability of the rumen microbiome to produce methane and/or to reduce methane production by a ruminant animal and/or to increase feed efficiency, milk production, and/or body weight or body composition of a ruminant animal. Methods for using Lacticaseibacillus rhamnosus strain HN001 or derivatives thereof and ruminant feed compositions comprising same are also provided.

What we claim is:

1. A method for inhibiting the growth of methane-producing bacteria and/or archaea in the forestomach of ruminant animals, wherein the method comprises administering to a ruminant animal an effective amount of Lacticaseibacillus rhamnosus strain HN001, AGAL deposit number NM97/09514 dated 18 Aug. 1997, or a derivative thereof.

2. A method for reducing ruminal methane production by a ruminant animal, wherein the method comprises administering to the animal an effective amount of Lacticaseibacillus rhamnosus strain HN001, AGAL deposit number NM97/09514 dated 18 Aug. 1997, or a derivative thereof.

3. A method for increasing feed efficiency in a ruminant animal, wherein the method comprises administering to the animal an effective amount of Lacticaseibacillus rhamnosus strain HN001, AGAL deposit number NM97/09514 dated 18 Aug. 1997, or a derivative thereof.

4. A method for reducing the ability of the rumen microbiome to produce methane, wherein the method comprises administering to the animal an effective amount of Lacticaseibacillus rhamnosus strain HN001, AGAL deposit number NM97/09514 dated 18 Aug. 1997, or a derivative thereof.

5. The method of any preceding claim, wherein the method inhibits the growth of hydrogenotrophic methanogens in the forestomach of the animal, preferably a methanogen from the genus Methanobrevibacter.

6. The method of any preceding claim, wherein L. rhamnosus HN001 or derivative thereof is administered in a composition that is a food, drink, food additive, drink additive, animal feed, animal feed additive, animal feed supplement, dietary supplement, carrier, vitamin or mineral premix, nutritional product, enteral feeding product, soluble, slurry, supplement, pharmaceutical, lick block, drench, tablet, capsule, pellet or intra-ruminal product, e.g., a bolus, or wherein the L. rhamnosus HN001 is encapsulated, for example in liposomes, microbubbles, microparticles or microcapsules.

7. The method of claim 6, wherein L. rhamnosus HN001 or derivative thereof is administered in drinking water, milk, milk powder, milk replacement, milk fortifier, whey, whey powder, Partial or Total Mixed Ration (TMR), corn, soybean, forage, grain, distiller's grain, sprouted grain, legumes, vitamins, amino acids, minerals, fibre, fodder, grass, hay, straw, silage, kernel, leaves, meal, solubles, slurries, supplements, mash feed, meal, fruit pulp, vegetable pulp, fruit or vegetable pomace, citrus meal, wheat shorts, corn cob meal, molasses, sucrose, maltodextrin, rice hulls, vermiculite, zeolites or crushed limestone.

8. The method of any preceding claim, wherein the method comprises administering to the animal L. rhamnosus HN001 in an amount of from 10⁴ to 10¹³ colony forming units per kilogram of dry weight carrier feed, from 10⁴ to 10¹⁰ colony forming units per kilogram of body weight of the animal per day, or from 10⁴ to 10¹³ colony forming units per day.

9. The method of claim 8, wherein the method comprises administering to the animal L. rhamnosus HN001 in an amount from 10⁸ to 10¹² colony forming units per kilogram of dry weight carrier feed, from 10⁵ to 10⁸ colony forming units per kilogram of body weight of the animal per day, or from 10⁶ to 10¹³ colony forming units per day.

10. The method of any preceding claim, wherein the derivative of L. rhamnosus HN001 is a cell lysate of the strain, a cell suspension of the strain, a metabolite of the strain, a culture supernatant of the strain, or killed L. rhamnosus HN001.

11. The method of any preceding claim, the method comprising further administering at least one microorganism of a different species or strain, a vaccine that inhibits methanogens or methanogenesis, and/or a natural or chemically-synthesised inhibitor of methanogenesis and/or methanogen inhibitor such as bromoform.

12. The method of any preceding claim, wherein the L. rhamnosus HN001 or derivative thereof is administered separately, simultaneously or sequentially with one or more agents selected from one or more prebiotics, one or more probiotics, one or most postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

13. The method of any one of claims 1 to 12, wherein the method enhances the growth or productivity of the ruminant animal.

14. The method of claim 13, wherein the method increases the yield of milk and/or milk components produced from the ruminant animal.

15. The method of claim 14, wherein the method increases the yield of milk fat, milk protein or milk solids in milk produced from the ruminant animal.

16. The method of any one of claims 1 to 15, wherein the method additionally improves the body weight and/or body composition of the ruminant animal.

17. The method of any preceding claim, wherein said ruminant animal is a bovine, goat, sheep, bison, yak, water buffalo, deer, camel, alpaca, llama, wildebeest, antelope, or nilgai.

18. The method of any preceding claim, wherein said ruminant animal are cattle or sheep.

19. The method of any preceding claim, wherein said ruminant animal are cattle.

20. The method of any preceding claim, wherein said ruminant animal is a lactating animal.

21. The method of any one of claims 1 to 19, wherein said ruminant animal is a pre-weaning animal, for example a calf or a lamb.

22. The method of any one of claims 1 to 19, wherein said ruminant animal is a post-weaning animal.

23. The method of any one of claims 1 to 19, wherein the L. rhamnosus HN001 is administered to the ruminant animal both prior to weaning and after weaning.

24. The method of claim 23, wherein the administering is to a pre-weaning animal and wherein the inhibition of the growth of methane-producing bacteria and/or archaea in the forestomach of the ruminant animals, the reduction of methane production by the ruminant animal, and/or the increased feed efficiency in the ruminant animal persists post-weaning.

25. The method of any preceding claim, wherein the inhibition of the growth of methane-producing bacteria and/or archaea in the forestomach of the ruminant animals, the reduction of methane production by the ruminant animal, and/or the increased feed efficiency in the ruminant animal persists for at least 2 days, 3 days, 5 days, 1 week, 2 weeks, 1 month, 2 months, 3 months, 4 months, 5 months, 6 months, 7 months 8 months, 9 months, 10 months, 11 months, 1 year, 2 years, 3 years, 4 years, 5 years, 6 years, or 7 years after the last administration of L. rhamnosus HN001.

26. The method of claim 25, wherein the inhibition of the growth of methane-producing bacteria and/or archaea in the forestomach of the ruminant animals, the reduction of methane production by the ruminant animal, and/or the increased feed efficiency in the ruminant animal persists for the life of the ruminant animal.

27. A ruminant feed composition for inhibiting the growth of methane-producing bacteria and/or archaea in the forestomach of ruminant animals, reducing the ability of the rumen microbiome to produce methane, reducing ruminal methane production by a ruminant animal, increasing feed efficiency in a ruminant animal, enhancing the growth and/or productivity in a ruminant animal, increasing the yield of milk and/or milk components produced from a ruminant animal, or improving the body weight and/or body composition of a ruminant animal, the feed composition comprising Lacticaseibacillus rhamnosus strain HN001 or a derivative thereof.

28. The ruminant feed composition of claim 27, wherein the feed composition a fermented yoghurt-style composition, and wherein the fermented yoghurt-style composition is formed through a process of growing L. rhamnosus HN001 using a milk-based carrier or a non-milk-based carrier.

29. The ruminant feed composition of claim 27, which is or comprises Partial or Total Mixed Ration (TMR), corn, soybean, forage, grain, distiller's grain, sprouted grain, legumes, fibre, fodder, grass, hay, straw, silage, kernel, leaves, meal, mash feed, lick block, or molasses.

30. The ruminant feed composition of any one of claims 27 to 29, further comprising at least one microorganism of a different species or strain, a vaccine that inhibits methanogens or methanogenesis, and/or a natural or chemically-synthesised inhibitor of methanogenesis and/or methanogen inhibitor such as bromoform.

31. The ruminant feed composition of any one of claims 27 to 30, further comprising one or more agents selected from one or more prebiotics, one or more probiotics, one or more postbiotics, one or more sources of dietary fibre, one or more galactooligosaccharides, one or more short chain galactooligosaccharides, one or more long chain galactooligosaccharides, one or more fructooligosaccharides, inulin, one or more galactans, one or more fructans, lactulose, or any mixture of any two or more thereof.

32. The ruminant feed composition of any one of claims 27 to 31, wherein the derivative of L. rhamnosus strain HN001 is a cell lysate of the strain, a cell suspension of the strain, a metabolite of the strain, a culture supernatant of the strain, or killed L. rhamnosus HN001.

33. A method for inhibiting the growth of methane-producing bacteria and/or archaea in the forestomach of a ruminant animal, said method comprising the step of administering to said animal a ruminant feed composition as claimed in any one of claims 27 to 32.

34. A method for reducing ruminal methane production by a ruminant animal, said method comprising the step of administering to said animal a ruminant feed composition as claimed in any one of claims 27 to 32.

35. A method for increasing feed efficiency in a ruminant animal, said method comprising the step of administering to said animal a ruminant feed composition as claimed in any one of claims 27 to 32.

36. A method for enhancing the growth and/or productivity in a ruminant animal, said method comprising the step of administering to said animal a ruminant feed composition as claimed in any one of claims 27 to 32.

37. A method for increasing the yield of milk and/or milk components produced from a ruminant animal, said method comprising the step of administering to said animal a ruminant feed composition as claimed in any one of claims 27 to 32.

38. A method for improving the body weight and/or body composition of a ruminant animal, said method comprising the step of administering to said animal a ruminant feed composition as claimed in any one of claims 27 to 32.

39. A method for reducing the ability of the rumen microbiome to produce methane in a ruminant animal, said method comprising the step of administering to said animal a ruminant feed composition as claimed in any one of claims 27 to 32.

40. Use of Lacticaseibacillus rhamnosus strain HN001, AGAL deposit number NM97/09514 dated 18 Aug. 1997, or a derivative thereof for the manufacture of a composition for inhibiting the growth of methane-producing bacteria and/or archaea in the forestomach of ruminant animals, reducing ruminal methane production by a ruminant animal, increasing feed efficiency in a ruminant animal, increasing the yield of milk and/or milk components produced from a ruminant animal, or improving the body weight and/or body composition of a ruminant animal.

41. Use according to claim 40, wherein the composition comprises a ruminant feed composition as claimed in any one of claims 27 to 32.

42. Lacticaseibacillus rhamnosus strain HN001, AGAL deposit number NM97/09514 dated 18 Aug. 1997, or a derivative thereof for use in inhibiting the growth of methane-producing bacteria and/or archaea in the forestomach of ruminant animals, reducing the ability of the rumen microbiome to produce methane, reducing ruminal methane production by a ruminant animal, increasing feed efficiency in a ruminant animal, increasing the yield of milk and/or milk components produced from a ruminant animal, or improving the body weight and/or body composition of a ruminant animal.
